(19) United States
(12) Patent Application Publication
(10) Pub. No.: US 2013/0045474 A1
(43) Pub. Date: Feb. 21, 2013

(54) DEVICES AND METHODS FOR DETECTING AND MONITORING HIV AND OTHER INFECTIONS AND DISEASES

(71) Applicants: Tomasz Rozmyslowicz, Williamstown, NJ (US); Glen N. Gaulton, Havertown, PA (US); Ryszard M. Lec, Philadelphia, PA (US)

(72) Inventors: Tomasz Rozmyslowicz, Williamstown, NJ (US); Glen N. Gaulton, Havertown, PA (US); Ryszard M. Lec, Philadelphia, PA (US)

(73) Assignees: Drexel University, Philadelphia, PA (US); The Trustees of the University of Pennsylvania, Philadelphia, PA (US)

(21) Appl. No.: 13/496,373

(22) PCT Filed: Sep. 23, 2010
(86) PCT No.: PCT/US10/49989

§ 371 Date: May 24, 2012

Related U.S. Application Data
(60) Provisional application No. 61/244,988, filed on Sep. 23, 2009.

(51) Int. Cl. G01N 29/02; C12M 1/34

(52) U.S. Cl. 435/5; 435/287.2

(57) ABSTRACT

Disclosed herein are bio-nanosensor devices and methods suitable for blood assays. The bio-nanosensors are based on thickness shear mode transducer capable of transmitting a shear wave into a biofluid adjacent to a bio-functionalized sensing interface of a piezoelectric crystal. The bio-functionalized sensing interface includes one or more antibodies and/or biomarker-specific ligands capable of sensing HIV. The disclosed bio-nanosensors are capable of defecting the presence of HIV virus at picogram sensitivities using no more than 10 μl of blood in less than 15 minutes.

### CROSS REFERENCE TO RELATED APPLICATIONS

This patent application claims the benefit of priority to U.S. Provisional Application Ser. No. 61/244,988, “DEVICES AND METHODS FOR DETECTING AND MONITORING HIV AND OTHER INFECTIONS AND DISEASES”, filed Sep. 23, 2009, the entirety of which is incorporated herein by reference for all purposes.

### TECHNICAL FIELD

The disclosed invention is in the field of biological sensors. The disclosed inventions are also in the field of detecting and monitoring infections, such as HIV.

### BACKGROUND

Following the introduction of highly active antiretroviral therapy (“HAART”), and recent improvements in its application and implementation, there has been a dramatic increase in the number of persons living with HIV infection and/or AIDS. In the U.S. and Canada alone, the number of adults over 50 with HIV/AIDS more than doubled in the last six years to an estimated 1.3 million infected people. The vast majority of these individuals require both continual access to anti-viral therapy and constant monitoring of HIV load to control disease.

Unfortunately, the current care strategies for patients with HIV infection face challenges associated with highly complex treatment paradigms that necessitate precise coordination in populations that are often subject to additional health considerations and medications. Such patients are often forced to endure a lower quality of life marked by the high possibility of treatment complications and diminished medical outcomes. As the number of persons infected with HIV is forecast to grow even more in the years to come, treatment strategies will have to significantly improve in order to better accommodate the desired paradigms of care. Accurate and sensitive diagnosis of the status of HIV infection is critical to this end; however, this must be cast in the context of multiple other biomarkers of health status linked to either HIV infection or the side effects of HAART—and this must all be accessible to individuals, who may be less mobile and/or less inclined to maintain regular clinic visits.

Existing HIV screening methods are limited by cost, timing, sensitivity and/or requirements for technical sophistication in delivery and assay conduct. Detection of HIV by antibody/immunological methods is available in over-the-counter kit form, but this method is not reliably sensitive until 4-6 weeks after exposure when the immune response to HIV is fully induced. Perhaps more importantly, this kit is intended to measure exposure to HIV: it is not suitable for direct measurement of HIV infection or of monitoring HIV infection levels over time, as serum antibodies to HIV are the source, not HIV itself. The p24 ELISA/SPR analysis can also be used to confirm infection and viral replication. This ELISA test (e.g., the Alliance HIV-1 p-24 ELISA kit—PerkinElmer, Boston, Mass.) directly detects internal HIV-1 protein p24, and works early after exposure. This test is reliable and sensitive, but must be performed in a certified laboratory with dedicated, sophisticated equipment to detect the SPR emissions. Molecular tests are also available to directly measure the presence of virus. These tests are based on analysis of virus DNA or RNA sequence in blood cells isolated from the patient. Specifically, HIV load within blood plasma may be measured using either: the NucliSens EasyQ HIV-1 v1.1 assay (Biomerieux, Durham, N.C.) that uses a nucleic acid sequence based amplification method; the Procleix HIV-1/HCV or Ultrio Assays in combination with HCV and/or 2nd HBV assays (Chiron/Novartis Corporation (Emeryville, Calif.) that utilize a branched DNA method, or the Roche Amplicor HIV-1 Monitor test (F. Hoffmann-La Roche Ltd, Basel, Switzerland) that utilizes PCR methods. The molecular tests measure HIV directly through the presence of the virus genome, however they require substantial amounts of blood (up to several ml) and a blood lab technician, the use of dedicated commercial laboratories to conduct molecular analysis, and they are accordingly quite expensive. Importantly, for the ELISA and molecular tests patients and/or health professionals must often wait up to two weeks for results since most local clinics send samples to outside specialty labs for analysis.

Accordingly, there is an urgent need to develop rapid, accurate, portable and inexpensive tests for HIV/AIDS patients to directly monitor virus infection levels and response to treatment, including in time HIV vaccines: let alone the ability to co-monitor other medically related conditions. Accordingly, new intervention methods are desperately needed toward the diagnosis, and coordination and simplification of HIV treatment for individuals who are either less mobile, less affluent or who reside in locations that are not contiguous with modern health care facilities. Nearly identical circumstances, and their linked restrictions, exist for a multitude of other infectious agents including viruses (e.g., Hepatitis C Virus), bacteria (e.g., Tubercles bacillus), fungi (e.g., Pneumocystis pneumonia), protozoans (e.g., Cryptosporidium) and parasites (e.g., Trichomonas vaginalis). The disclosed inventions are aimed to fill these needs.

### SUMMARY

The disclosed inventions pertain to bio-nanosensor diagnostic/treatment monitoring devices and to methods of using such bio-nanosensors. The bio-nanosensors disclosed herein are capable of providing important medical information regarding disease status on a real-time basis to health workers as well as patients in the home setting. In particular, these inventions enable an HIV/AIDS management system that is capable of efficient and effective monitoring and treatment of HIV infection and its complications.

The bio-nanosensors disclosed herein are small, portable, hand-held nano-detection and monitoring devices that are capable of providing real-time measurement of HIV infection levels in biofluids such as blood, urine or saliva. Additional related health condition information on patients can also be measured simultaneously. In addition to the direct measurement of HIV levels, this health profile can include immune function and metabolic status thus enabling evaluation of total response to treatment. As the devices are both mobile and self-contained it may be used in the home and/or other point-of-contact settings, and is thus ideal for the diagnosis, monitoring and care of individuals with restricted mobility or access to health care clinics, such as the elderly. The specific profile of an individual can be determined by a set of applied biomarkers used in a disposable bio-nanosensor of the device. The results can be displayed on a hand-held screen and can be accompanied by a standard medical diagnosis. The data are digitally stored in the device enabling the patient and, where relevant, health care supervisor to conduct disease tracking over time.

The disclosed bio-nanosensors and methods of their use address a critical and currently unmet need in HIV detection: enabling direct, highly sensitive measurement of HIV virus levels by the individual in a home-setting, obviating the need for repeated clinical visits which may be costly and/or impractical for certain individuals. In addition, by coupling HIV detection to other biomarkers of health status the bio-nanosensor device provides more timely and comprehensive medical information related to the general health status of the individual. As a result the present invention advances and enables new point-of-care services and home-based medical procedures, thereby improving HIV/AIDS patient health conditions and quality of life.

Suitable bio-nanosensors comprise a vibrating electromechanical structure characterized whereby an applied alternating electrical voltage induces an oscillating mechanical strain over a broad frequency range, and whereby the electromechanical structure is capable of transmitting a mechanical wave into a bio fluid medium adjacent to a bio-functionalized sensing interface of the structure to produce a variation to the operating parameters of the vibrating sensing structures such as a resonant acoustic wave frequency, resonant attenuation, quality factor, slope, phase characteristic and those changes are measurable by said biosensor detecting system; wherein the biofluid contacting surface comprises one or more biomarkers indicative of a disease or physiological state; a fluidic chamber capable of containing said biofluid, the fluidic chamber comprising one or more fluidic conduits capable of fluidicly communicating at least one or more fluids; fluidic body fluids (blood, saliva, urine) sample collection structures; and one or more electrical leads in electrical communication with one or more electrodes mounted directly adjacent to said electromechanical structural and said biofluid contacting surface.

In particular, the bio-nanosensors comprise a multi-resonant thickness shear mode transducer comprising a piezoelectric crystal/polycrystal characterized whereby an applied alternating electrical voltage induces an oscillating shear mechanical strain over a broad frequency range, and whereby the thickness shear mode transducer is capable of producing a standing acoustic wave within the piezoelectric vibrating structure, the thickness shear mode transducer being capable of transmitting a shear wave into a bio fluid medium adjacent to a bio-functionalized sensing interface of the piezoelectric vibrating structure to give change to the operating parameters of the structure and the change measurable by said biosensor device; wherein the biofluid contacting surface comprises one or more biomarkers indicative of a disease state; a fluidic chamber capable of containing said biofluid, the fluidic chamber comprising one or more fluidic conduits capable of fluidicly communicating at least one fluid; and one or more electrical leads in electrical communication with one or more electrodes mounted directly adjacent to said piezoelectric quartz crystal and said biofluid contacting surface.

Suitable piezoelectric-based bio-nanosensors are capable of rapid detection of HIV in blood based on the presence of gp120, and independently p24. Suitable piezoelectric bio-nanosensor assays are capable of detecting clinically relevant HIV concentrations based on direct measurement of gp120 and p24 in blood. Accordingly, the bio-nanosensor devices of the present invention are capable of providing a sensitive, rapid, inexpensive and portable functionality for detecting the presence of HIV and other serum markers with an actual detection time of less than 15 minutes while using no more than 10 μl of blood.

Bio-nanosensors comprising multiple sensing biomarkers are also provided by the present invention. A number of biomarkers can also be integrated on the bio-nanosensors with or without the HIV-detection component. In the instance of HIV-1 infection this enables co-monitoring for any of a number of secondary diseases known to afflict HIV infected patients. For example biomarkers of viral infections (Cytomegalovirus, hepatitis, herpes simplex, herpes zoster, human papillomavirus, Epstein-Barr virus, Influenza virus, West Nile virus, SARS, human T-leukemia viruses, etc), bacterial infections (Mycobacterium avium complex, salmonellosis, syphilis, tubecolosis, etc), fungal infections (aspergillosis, candidiasis, coccidioidomycosis, cryptococcosis, histoplasmosis, etc), protozoal infections (cryptosporidiosis, isosporiasis, microsporidiosis, pneumocystis carinii, toxoplasmosis, malaria, etc), and other diseases e.g.: diseases of heart, kidney, liver, central and peripheral nervous system, metabolic diseases (diabetes, etc) may be integrated on the bio-nanosensor platform.

In addition to measuring gp120 and p24 biomarkers for HIV and similar molecular components of other infectious agents, the bio-nanosensors are also readily adapted to include physiological biomarkers immobilized at the bio-functionalized sensing interface that are capable of measuring, for example, CD4, insulin, C-peptide, IL-6 and HbA_{1C}. Such bio-nanosensor assays are capable of providing a specific health profile on the health conditions of HIV/AIDS patients and can be prepared on a single substrate. Bio-nanosensor are readily optimized by determining the operating conditions such as: size of electrode, packing density, optimum dimensions, acoustic mode of interaction, software and hardware development for monitoring multiple sensors and signal processing.

Accordingly, the present inventions also provide bio-nanosensor systems capable of simultaneously detecting a panel of biomarkers relevant to the health status of individuals infected with HIV as well as other microbial and parasitic agents. Bio-nanosensor systems can measure the concentration of one or more physiological biomarkers comprising elements such as gp120, p24, CD4, insulin, C-peptide, IL-6, HbA_{1C} using suitable antibodies for each of these biomarkers linked to the biofluid contacting surface.

Using several different biomarkers, the bio-nanosensor is capable of providing a comprehensive health profile of the individual. In the instance of HIV, one or both of two HIV-1 markers, gp120 and p24, immobilized at the surface of the biofluid contacting surface, provide information on the level of infection and response to treatment. Knowing that age-related difference in response to HAART have been observed with respect to CD4 response and viral clearance, the bio-nanosensors are capable of measuring CD4 levels which will allow health professionals to utilize this bio-nanosensor for more careful monitoring of the treatment process. The bio-nanosensors are capable of providing real-time feedback for optimizing treatment to HIV and other diseases. As it is easy to use by an untrained person, suitable bio-nanosensors can be in the form of a hand-held pencil like device. For monitoring certain patients, such as the elderly, the bio-nanosensors can further couple biomarkers of infection, such as HIV-1, to those for other diseases such as diabetes. In this instance a bio-nanosensor can be configured for example to measure plasma insulin, C-peptide, HbA1c and IL-6, which measurements can be coupled to the measurement of serum glucose by existing glucose tests to obtain a patient's full profile.

### BRIEF DESCRIPTION OF THE DRAWINGS

The foregoing and other aspects of the present invention is apparent from the following detailed description of the invention when considered in conjunction with the accompanying drawings. For the purpose of illustrating the invention, there is shown in the drawings embodiments that are presently preferred, it being understood, however, that the invention is not limited to the specific instrumentalities disclosed. The drawings are not necessarily drawn to scale. In the drawings:

FIG. 1 illustrates a conceptual model for a TSM sensor (sensor operating at the fundamental frequency and higher;

FIG. 2 illustrates (a) a typical frequency-dependent response curve for the bio-nanosensor in the vicinity of the fundamental resonant frequency; t1, t2 and t3 represent progressively loaded sensors; the corresponding frequency (f) and amplitude (R) values are both shown to decrease with time;

FIG. 3 illustrates (A) an embodiment of a bio-nanosensor device of the present invention—a prototypical bio-nanosensor chamber (TSM is enclosed in the center window); (B) the sensor resonant frequency change following steps of the immobilization process and sensor exposure to gp120;

FIG. 4 illustrates the frequency response following the addition of gp120 to the bio-nanosensor coated with anti-gp120;

FIG. 5 illustrates the bio-nanosensor resonant frequency change in response to HIV Negative Control-NL43 virus. Positive Control-R3A virus pseudotyped with R5/X4 ENV;

FIG. 6 illustrates (a) an embodiment of the bio-nanosensor device of the present invention—a single TSM sensor, and (b) an embodiment of the bio-nanosensor device of the present invention comprising a plurality of TSM sensors—a TSM sensor array; (c) an image of 5 and 100 MHz TSM sensors;

FIG. 7 illustrates an embodiment of a bio-nanosensor device of the present invention—HIV/AIDS Biochip Test Strip comprising (A) a disposable test strip complete with packaged reagents and electrical connections, and (B) an array of a plurality of TSM sensors and display module;

FIG. 8 illustrates a suitable HFPB electronic measurement system;

FIG. 9 illustrates an anticipated distribution (health profile) of HIV/AIDS patients with insulin resistance and diabetes;

FIG. 10 illustrates the steps for fabricating an embodiment of a bio-nanosensor according to the present invention;

FIG. 11 illustrates the bio-nanosensor profile results using the devices and methods of the present invention that can be attained from HIV/AIDS patients with anemia and kidney involvement;

FIG. 12 illustrates the bio-nanosensor profile results using the devices and methods of the present invention that can be attained from HIV/AIDS patients with liver involvement I;

FIG. 13 illustrates the bio-nanosensor profile results using the devices and methods of the present invention that can be attained from HIV/AIDS patients with liver involvement II;

FIG. 14 illustrates the bio-nanosensor profile results using the devices and methods of the present invention that can be attained from HIV/AIDS patients with secondary viral infection I;

FIG. 15 illustrates the bio-nanosensor profile results using the devices and methods of the present invention that can be attained from HIV/AIDS patients with secondary viral infection II;

FIG. 16 illustrates the response of a BNS to a negative and positive HIV blood samples; and

FIG. 17 illustrates the sensitivity characteristics of a BNS to various concentrations of virus particles.

### DETAILED DESCRIPTION AND ILLUSTRATIVE EMBODIMENTS

The present subject matter may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it is understood that the particular value forms another embodiment. All ranges are inclusive and combinable.

Piezoelectric High Frequency Bio-Nanosensors. The bio-nanosensor devices of the present invention comprise a vibrating electro-mechanical structures such as multi-well array of radial, flexural and thickness shear mode transducers, a bio-functionalized sensing interface for contacting a biofluid, such as blood, a fluidic chamber capable of containing the biofluid, and one or more electrical leads in electrical communication with the thickness shear mode transducer. In some embodiments, as further described herein, the bio-nanosensor devices are capable of detecting the presence of HIV viruses in no more than 10 μl of blood in less than 15 minutes.

Suitable thickness shear mode transducers comprise a piezoelectric crystal characterized whereby an applied alternating electrical voltage induces an oscillating shear mechanical strain over a broad frequency range. The thickness shear mode transducers are capable of producing a standing acoustic wave within a piezoelectric crystal when actuated by an electrical signal. Accordingly, the thickness shear mode transducers are capable of transmitting a shear wave into a biofluid adjacent to a bio-functionalized sensing interface of the piezoelectric crystal. This shear wave produces measurable changes in the operation parameters of the sensor such as resonant acoustic wave frequency change, or amplitude, or phase, or the slope of those or quality factor.

The bio-functionalized sensing interfaces can comprise one or more antibodies or ligands that are capable of specific binding to a biomarker for HIV (e.g., HIV-1 and HIV-2) or directly to an HIV virus, or both, as well as other important infectious agents or physiological biomarkers, which may be associated with HIV infection. These include infectious agents such as: viral infections (Cytomegalovirus, hepatitis, herpes simplex, herpes zoster, human papillomavirus, Epstein-Barr virus, Influenza virus, West Nile virus, SARS, human T-leukemia viruses, etc), bacterial infections (Mycobacterium avium complex, salmonellosis, syphilis, tubecolosis, etc), fungal infections (aspergillosis, candidiasis, coccidioidomycosis, cryptococcosis, histoplasmosis, etc), protozoal infections (cryptosporidiosis, isosporiasis, microsporidiosis, pneumocystis carinii, toxoplasmosis, malaria, etc), and parasitic infections (trichomonas, ascaris, opisthorchis etc). These also include physiological biomarkers of immune status, not limited to CD4, as well as other carbohydrates, lipids, and proteins contained in body fluids. Suitable antibodies capable of specific binding to a biomarker for HIV that can be used for detecting HIV include monoclonal antibodies against Env-anti gp120, anti gp41, anti-gp160, anti-V3, anti Gag-anti p24, anti Nef, anti-Pol and Rev-anti RT, anti-IN, anti-Tat, anti-Vif, anti-Vpx, anti-p27 and also anti-CD4 and polyclonal antibodies: anti-Env-anti gp120, anti-gp160, anti-Gag-anti p17, anti p24, anti-Nef, anti-Pol-anti-protease, anti-integrase, anti-Tat, anti Vpr, anti-Vif, anti-Vpu, anti-Vpx, anti-CD4, anti-gp120, anti-p24 and anti-CD4, and any combination thereof. The antibodies are immobilized at the bio-functionalized sensing interface using any of a variety of methods as described further below. In some embodiments, the bio-nanosensor device of claim A, further comprising one or more additional biomarker-sensing ligands specific to one or more biomarkers for monitoring the presence of one or more additional infectious agents or disease states other than HIV/AIDS, the biomarker-sensing ligands immobilized at the bio-functionalized sensing interface.

Suitable bio-functionalized sensing interfaces are selected to avoid non-specific binding of other chemical and molecular species found in the biological fluids. As bio-nanosensors can be prepared label-free to detect the physical presence of chemical and/or molecular species adsorbed on the transducer surface, including the bio-functionalized sensing interface, nonspecific binding is minimized. Non-specific binding can be minimized using any of a variety of blocking techniques (as described above), or for example, using self assembled monolayers (SAM).

One or more fluidic chambers are included in the bio-nanosensors. The fluidic chambers are capable of containing a biofluid, the fluidic chamber comprising one or more fluidic conduits capable of fluidically communicating at least one fluid from a specimen to the bio-functionalized sensing interfaces. Fluidic conduits can also be included for fluidically communicating at least one fluid comprising a washing fluid, a blocking agent (e.g., BSA), a buffer, a biomarker, an antibody, a biofluid, an antigen, a coupling agent, a wetting agent, a cleaning agent, or any combination thereof.

The bio-nanosensors are supplied with one or more electrical leads to actuate the thickness shear mode transducers. The electrical leads are suitably in electrical communication with one or more electrodes mounted adjacent to a piezoelectric crystal, a bio-functionalized sensing interface, and a power source and/or signal generator. The application of an electrical signal to the electrodes gives rise to an oscillation of the piezoelectric crystal, which will oscillate at different frequencies depending on the mass and shape of the crystal, the viscosity of the medium in which it sits, as well as the presence of adsorbed molecular entities on its surface.

The bio-nanosensor devices may also include a plurality of thickness shear mode transducers for detecting and/or measuring the concentration of more than one type of biomarker. In these embodiments, at least one of said thickness shear mode transducers can comprise an antibody or biomarker-sensing ligand immobilized at its bio-functionalized sensing interface that is different from at least one other of the biomarker-sensing ligands of one or more other thickness shear mode transducers.

Micro-electronic and micro-mechanical fabrication technologies can be used for fabricating the bio-nanosensors of the present invention. In view of the availability of such small scale manufacturing technologies, piezoelectric high frequency bio-nanosensors can be used for realizing a variety of sensing devices that exhibit high sensitivity, small size and portability, fast responses, ruggedness and robustness, high accuracy, compatibility with Integrated Circuit (IC), MEMS and NEMS technologies, excellent aging characteristics and the capability of measuring multiple quantities in one sensor package. Bio-nanosensors based on this technology can be produced using known photolithographic techniques. The application of combined cleaning, photolithography, etching and deposition processes can be used in the manufacture of quartz resonators with higher resonant frequencies, up to a few hundred MHz and smaller diameters. Flat, smooth piezoelectric membranes are obtained, which are characterized has having good sidewall profiles to accomplish low noise, low loss and high Q-factor. Defect density of the piezoelectric membranes are also very low as a result of the thinness of the membranes. Suitable membrane thickness can range from several hundred microns to single microns. Membrane surface roughness can be varied too and generally scales from the nanometer range for optical quality to several microns.

In one embodiment there is provided a piezoelectric high frequency bio-nanosensor assay that includes eight sensing elements. Testing is carried out for each patient sample carrying target health conditions. The response of each assay is qualitatively compared to the values obtained from ELISA analysis, in particular regarding sensitivity and specificity.

Bio-nanosensors can be fabricated with a wide range of electrode geometries. Electrode dimensions can be in the range of from microns to millimeters. The electrodes could be of different size and shapes. For examples, electrodes can vary from 10, 20, 40, 60, 80, 100, or even hundreds of microns in characteristic dimension, and even up to several millimeters. Electrode shapes can vary and include circular, rectangular, ellipsoidal, oval, interdigital, and the like. Suitable distance is maintained between electrodes for increasing mass sensitivity or eliminating interference between TSM sensors, or both.

Suitable bio-nanosensors can be small (e.g., 1 cm×0.5 cm×0.5 cm) solid-state devices with disk, plate or prism shapes that have a system of metal electrodes used for interfacing a sensor with electronic circuits. Many types of piezoelectric sensors can be used in the present invention. Suitable piezoelectric sensors include a Multi-resonant Thickness Shear Mode (MTSM) Resonator, an acoustic plate mode (APM) device and a Surface Skimming Bulk Wave (SSBW) device. Each of these piezoelectric sensors are capable of generating pure shear motion and can be used for fluid sensing. Preferably, the bio-nanosensors of the present invention use TSM-based piezoelectric sensors, which sensors are fully described in WO 2007/040566, “Method and Apparatus for Interfacial Sensing” by Ryszard M. Lec, corresponding to U.S. National Stage patent application Ser. No. 11/719,895, filed Feb. 21, 2008, the entirety of which is incorporated by reference thereto.

Typical operating parameters of MTSMs include the operational frequency, the dynamic range and the noise level. The operational frequency of the TSM is dependent on the membrane thickness of the sensor. The dynamic range and the noise level are determined by the Q-factor of the TSM, which in turn is affected by the roughness, the flatness, and the low level of defects in the membrane. As a consequence, a well-controlled microfabrication processes are typically used to meet those conditions. Accordingly, bio-nanosensors can be fabricated using a suitable integrated circuit (IC) microfabrication processes. For example, piezoelectric materials such as quartz) is cut and polished to the required thickness and shape. Other piezoelectric materials can also be used, such as quartz, tourmaline, lithium tantalite, polyvinylidene fluoride, lanthanum gallium silicate, potassium sodium tartrate, ceramics with perovskite tungsten-bronze structures such as BaTiO₃, KNbO₃, Ba₂NaNb₅O₅, LiNbO₃, SrTiO₃, Pb(ZrTi)O₃, Pb₂KNb₅O₁₅, LiTaO₃, BiFeO₃, NaₓWO₃, as well as composite piezoelectric structures comprising piezoelectric materials such zinc oxide, aluminum nitrite, or a sol-gel derived PZT (lead zirconate titanate) thin films with various Zr/Ti ratios prepared on various sensing substrates. The masks for the given electrode pattern are developed and the metal electrodes can be either RF sputtered or made photolithographically. High frequency sensors suitable for operation above 50 MHz can be made using additionally a combination of reactive ion etching (RIE) and chemical wet etching techniques. Electrical connections can be made using any suitable techniques such as microprinting, electroplating and ultrasonic bonding.

Suitable piezoelectric substrates are characterized as having sufficient mechanical stability for handling. Sensing regions on the substrates can be provided using chemical etching to thin down the substrate to a desired thicknesses to give rise to suitable thin membranes with thick mechanically stable outer areas. A combination of wet/dry etching techniques can also be used as an alternative method to give rise to good step coverage, fewer defects, as well as flat and smooth surfaces.

Suitable piezoelectric high frequency sensors are label-free, small, rapid, inexpensive, portable and simple to use, and hence, are well suited for applications in analytical labs as well in point-of-care settings. Label-free sensing technique enables a rapid, simple and inexpensive detection of the target molecules (e.g., proteins, bacteria, cells, etc.). Oftentimes a single sensing step can be employed. The sensitivity of the bio-nanosensors is on the order of nanograms to picograms of mass detected on the sensor surface, and the detection time is in the range of from about 10 minutes to about 20 minutes. Bio-nanosensors can typically analyze the biological interface at a depth that is on the order of tens to hundreds of nanometers. Piezoelectric sensors, which function as resonant electromechanical structures, can be excited at their fundamental and harmonic frequencies which give them capabilities to generate acoustic waves having different penetration depths. This provides them the capability of “slicing” biological interfaces simultaneously at different depths, thus improving piezosensor selectivity, sensitivity, reliability, and confidence. Additionally, data obtained from multiple-frequency sensor responses, via appropriate signal processing, allow extraction of unique features of the bio-nanosensor response, thus provide an opportunity to simultaneously detect several targeted analytes from a single measurement.

The bio-nanosensor in some preferred embodiments is a portable, inexpensive and simple to use diagnostic test for early direct detection of HIV. The bio-nanosensor can be used for HIV diagnosis following potential exposure, for the tracking of virus levels in infected individuals, and in a related manner, for the management of HIV/AIDS disease progression in patients undergoing anti-HIV therapies such as HAART. As further described below, devices according to the present invention have been developed and tested using HIV biomarkers gp120 and p24 immobilized at a bio-functionalized sensing interface using a label-free piezoelectric bio-nanosensor.

Some bio-nanosensors of the present invention are capable of monitoring the kinetics of the target detection (e.g., interaction between the antibody and virus protein). Such monitoring is capable of comparing kinetics between reactions to enable quality control of the assay processes. The consistent reliability and sensitivity of the bio-nanosensor devices made according to the present invention enables the quantification of multiple protein interactions in patient blood samples accurately and rapidly. In this regard the bio-nanosensor devices and methods of the present invention can be used as small, portable HIV detection devices that provide an important breakthrough in quick diagnosis of HIV infection as well as in HIV treatment monitoring.

Methods of Detecting HIV Using Bio-Nanosensors. The methods of determining the presence of HIV virus in a biofluid include the steps of contacting a biofluid suspected of comprising HIV to a bio-functionalized sensing interface adjacent to a piezoelectric crystal, and inducing an oscillating shear mechanical strain to the piezoelectric crystal to give rise to a shear wave being transmitted into the biofluid adjacent to the bio-functionalized sensing interface. The frequency of the standing acoustic wave of the piezoelectric crystal is measured, which frequency is correlated to the presence of HIV virus in the biofluid. The bio-nanosensors described supra can be readily used in these methods. For example, any of the antibodies or biomarker-specific ligands specific for HIV can be immobilized to the bio-functionalized sensing interface, examples of which antibodies include anti-gp120 and anti-p24. The methods can be carried out using no more than 10 μl of blood specimens from patients to detect the presence of HIV virus in less than 15 minutes. Other biofluids (e.g., bodily fluids) can be tested as well, such as amniotic fluid, whole blood, blood plasma, blood serum, breast milk mucus (including nasal drainage and phlegm), pleural fluid, saliva semen, spinal fluid, sweat, tears, urine, vaginal secretion, vomit breast milk, and the like. The bio-functionalized sensing interface may further include one or more additional biomarker-sensing ligands specific to one or more biomarkers for monitoring the presence of one or more additional disease states other than HIV/AIDS. Suitable biomarker-sensing ligands, as described above, are immobilized at the bio-functionalized sensing interface.

The methods of the present invention may include additional steps for incorporating a control fluid in the testing protocol, which may be used for determining absolute or relative concentration of a virus or antigen in the biofluid. These methods further include the steps of contacting a control fluid not comprising a biomarker for the HIV virus, to the bio-functionalized sensing interface and inducing an oscillating shear mechanical strain of the piezoelectric crystal to give rise to a shear wave being transmitted into the control fluid adjacent to the bio-functionalized sensing interface of the piezoelectric crystal. The frequency of a standing acoustic wave of the piezoelectric crystal arising from the shear wave being transmitted into the control fluid is measured, and the difference between the frequency of the standing acoustic wave measured with the control fluid to the frequency of the standing acoustic wave measured with the biofluid is correlated to the presence of, relative concentration of, or absolute concentration of HIV virus in the biofluid.

Biofluids are typically contacted with the bio-functionalized sensing interface in a fluidic chamber. Suitable fluidic chambers include one or more fluidic conduits capable of fluidicly communicating at least one or more of the following fluids into the fluidic chamber: a washing fluid, a blocking agent, a buffer, a biomarker, an antibody, a biofluid, an antigen, a coupling agent, a wetting agent, a cleaning agent.

For detecting and/or monitoring more than one biomarker and/or disease state in a patient, a biofluid can be contacted to a plurality of bio-functionalized sensing interfaces, each biofluid contacting surface comprising an antibody or biomarker-sensing ligand attached thereto. In this embodiment, the antibodies or biomarker-sensing ligands immobilized at one of the bio-functionalized sensing interfaces is different than the antibodies or biomarker-sensing ligands immobilized at one or more of the other bio-functionalized sensing interfaces. Although some of the ligands can be the same on the interfaces, some embodiments also provide that each of the antibodies or biomarker-sensing ligands immobilized at each of the bio-functionalized sensing interfaces can be different too.

Monitoring the Progress of Therapy or Prevention. The present invention also provides for methods for monitoring the progress of therapy, such as HAART, of a patient having HIV virus and of prevention in the response to vaccines whether infected or not. In these methods, a biofluid specimen is obtained from an individual and contacted to a bio-functionalized sensing interface comprising one or more of the following antibodies: anti-gp120, anti-p24, anti gp41, anti-gp160, ati-V3, anti Gag-anti p24, anti Nef, anti-Pol and Rev-anti RT, anti-IN, anti-Tat, anti-Vif, anti-Vpx, anti-p27, anti p17, anti-Nef, anti-Pol-anti-protease, anti-integrase anti-Vpr, anti-Vpu, anti-CD4, and any combination there of. The antibodies are immobilized at the bio-functionalized sensing interface so that the presence of gp12 or p24 present in the individual's biofluid will bind to the interface, thereby changing the frequency of oscillation of a piezoelectric crystal coupled to the bio-functionalized sensing interface. In these methods, an oscillating shear mechanical strain of the piezoelectric crystal is induced using a suitable electrical signal to give rise to a shear wave being transmitted into the biofluid adjacent to the bio-functionalized sensing interface of the piezoelectric crystal. The frequency of the standing acoustic wave of the piezoelectric crystal is measured and correlated to the frequency of the standing acoustic wave to the concentration of HIV virus in the biofluid specimen to determine the progress of therapy.

Operation of Bio-Nanosensor Devices. Suitable bio-nanosensor devices can be used to measure gp120 and p24 protein concentrations in blood following their binding to the sensor surface. The concentrations of gp120 and p24 in blood relate directly to the level of virus infection. As described in detail below, the presence of these virus proteins is determined by attaching anti-gp120 or anti-p24 antibodies to the sensor surface at a bio-functionalized sensing interface, then after washing, virus proteins are added—either in isolated form or incorporated within virus particles—and the level of protein binding is measured by changes in the resonant frequency of the bio-nanosensor.

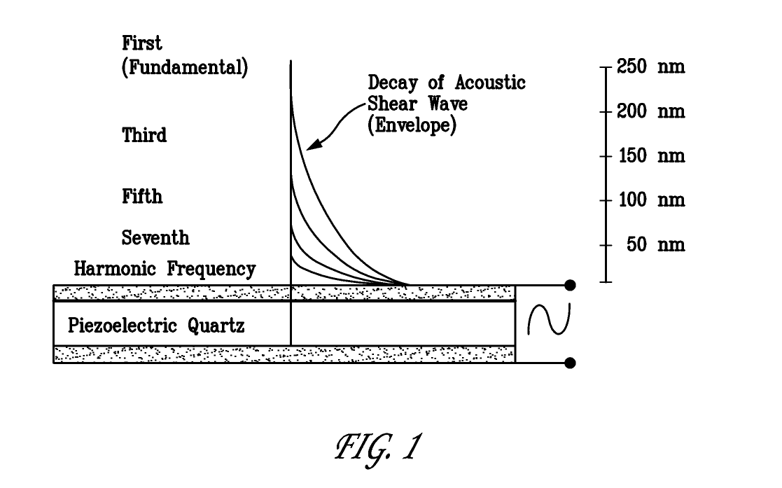

One element of the bio-nanosensor assay is a thickness shear mode (TSM) sensor that possesses the property whereby an applied electrical voltage induces a shear mechanical strain, over a broad frequency range. By exciting a TSM sensor with an alternating voltage, standing acoustic waves are produced within the sensor. The TSM sensor behaves as a highly sensitive electromechanical resonator, transmitting a shear wave into the liquid medium. This configuration can be represented as a coupled resonant system, the properties of which depend on the properties of the sensor, the medium, and the interface at the sensor/medium boundary. The shear wave penetrates a liquid over a very short distance, on the order of tens to hundreds of nanometers, and the influence of the boundary (interfacial) conditions on the behavior of the sensor is very strong. A shear acoustic wave decays rapidly with the rate determined by the penetration depth factor, which is proportional to liquid viscosity and inversely proportional to liquid density and the frequency of the wave. Therefore, by changing the frequency, one can control the distance at which the wave probes the sensor-liquid interface. FIG. 1 illustrates a conceptual model for a TSM sensor (sensor operating at the fundamental frequency and higher. For example, at 5 MHz in phosphate buffer saline (PBS), the depth of penetration is about 280 nanometers, and at 500 MHz is only 26 nanometers. If the frequency increases, then the depth of penetration decreases. Suitable bio-nanosensors operate in the range of from about single MHz to about several hundreds MHz. The multiresonant operation of the bio-nanosensor allows controlling the depth from the sensor response being collected; i.e., this process essentially probes individual slices of the fluid medium, which substantially improves the bio-nanosensor performance in term of sensitivity, selectivity and resolution.

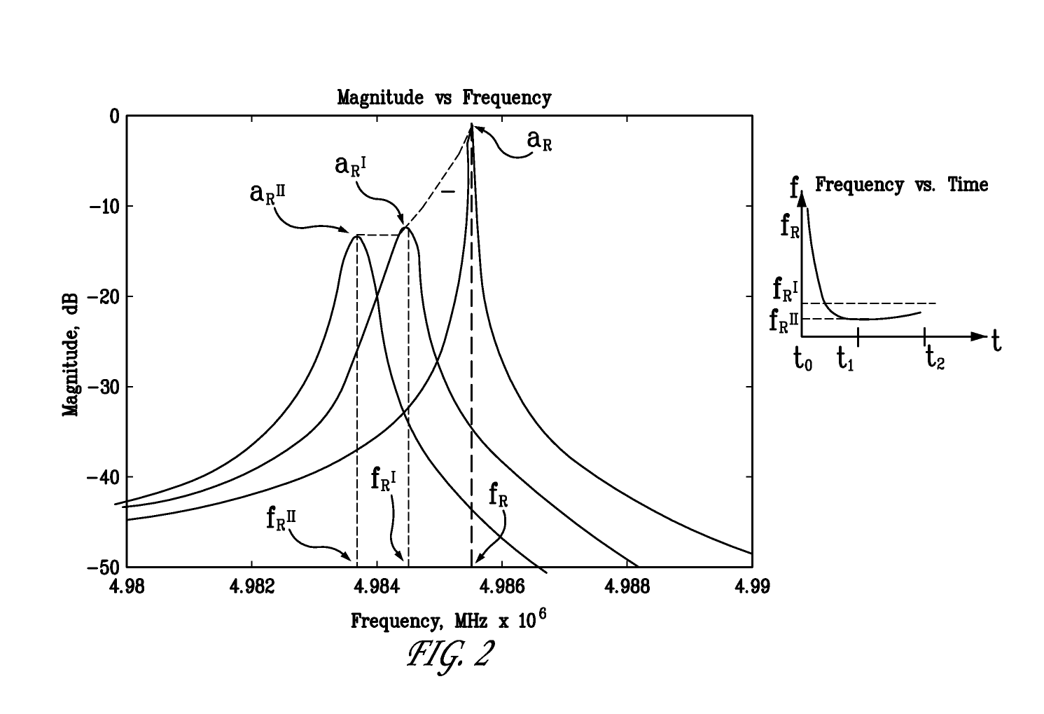

A typical electrical response of the TSM sensor, measured by the electronic detection system, in the vicinity of operating frequency range and the change in the frequency as a function of time are given in FIG. 2, respectively. This figure illustrates (a) a typical frequency-dependent response curve for the bio-nanosensor in the vicinity of the fundamental resonant frequency; t₁, t₂ and t₃ represent progressively loaded sensors; the corresponding frequency (f) and amplitude (R) values are both shown to decrease with time. The magnitude of the response, the S₂₁ scattering parameter, is defined as

|S₂₁|=20 log(100/(100+Zₜ)),

and Zₜ=total electromechanical impedance of the TSM sensor that is a function of the liquid loading. When the bio-nanosensor is loaded with a biological media, the sensor response S21 will exhibit a shift in its resonant frequency and a decrease in its magnitude. These changes can be correlated with the mass accumulation on the sensor interface because of the binding between one or more antibodies and antigens. Depending on the antibody-antigen interactions at the sensor surface-medium interface (i.e., the bio-functionalized sensing interface), a positive and/or negative shift results in the frequency response, which frequency response is readily observed.

Bio-nanosensors according to the present invention may also be operated using paired bio-nanosensors and digital readout components. The bio-nanosensors can operate at the nano-scale in terms of both detection sensitivity and size: specificity is linked to the use of antibody and/or defined ligand binding capabilities. The bio-nanosensor device described herein are capable of detecting nanogram and picogram quantities of the HIV proteins gp120 and p24, and of intact HIV-1 virus particles that contain ENV protein. Picogram scale sensitivity is useful for the desired sensitivity in whole blood/serum measurements.

The measured signals of the bio-nanosensors carry information related to the kinetics of biological interactions that can be used by one of ordinary skill in the art with the benefit of this specification in hand, is able to even further improve the selectivity and the confidence level of the bio-nanosensors and test methods.

Bio-nanosensor devices comprise sensor, electronics and software, all of which can be optimized by one of ordinary skill in the art to improve sensitivity and specificity. Similarly, further improvements to the immobilization protocols for attaching antibodies and/or other types of biomarker-sensing ligands to give rise to even more robust and stable bio-functionalized sensing interfaces.

### EXAMPLES AND OTHER ILLUSTRATIVE EMBODIMENTS

General Methods Used

Antibodies, Biomarker-specific ligands and Immobilization. Antibody, ligand proteins or other binding agents can be immobilized at the bio-functionalized sensing interface using any suitable protein-surface immobilization protocol including, for example, direct adsorption and covalent binding methods. Antibodies, biomarker-specific ligands and the like can be immobilized at the bio-functionalized sensing interface using one or more procedures. Suitable immobilization procedures include direct adsorption and covalent bonding, as well as self assembling of monolayers, followed by anchoring, as set forth below.

Preparation of Sensor Surfaces for Immobilizing Antibodies and Biomarker-Specific Ligands. Electrodes are preferably composed of an inert and biocompatible metal such as gold or platinum which are capable of immobilizing an antibody or biomarker-specific ligand. Gold electrode surfaces of suitable TSM sensors can be cleaned using Piranha solution (one part of 30% H₂O₂ in three parts H₂SO₄). After 2 min exposure time, the sensor is rinsed with distilled water. The surface is dried in a stream of nitrogen gas.

Direct Adsorption of Proteins (Antibodies). The cleaned TSM sensor is immersed in phosphate buffer saline (PBS). After the frequency and magnitude responses are stabilized, an aliquot (5 μl) of the capture antibody (anti-gp120, anti-p24; anti-CD4) is introduced on sensor surface. The frequency and magnitude changes due to the adsorption of the antibodies on the surface of electrodes is monitored as a function of time. The experiment is performed until the equilibrium is achieved. The concentration of the antibody solution is chosen to provide a full surface coverage of the sensor surface.

Self assembled monolayer (SAM): SAM methods using thiols are useful for their ability to readily adsorb and organize on gold surfaces and remain stable for extended periods of time. Once the thiol is anchored to the substrate, a peptide bond can be formed between an acid group on the thiol and an amino-terminus of the protein of choice using carbodiimide chemistry. For the antibody attachment on a gold surface using a SAM, the preparation method mentioned above can be used to ensure a clean surface. For example, TSM sensors are placed in absolute ethanol containing 2.5 mM 11-Mercaptoundecanoic Acid (MUA) and 7.5 mM 1-Decanethiol (NDt) for three hours at room temperature. After rinsing in absolute ethanol and drying under nitrogen gas, the thiolated substrates may be stored or used right away. To conjugate IgG on the thiolated surface of the transducer, a solution of 10 mM N-3-Dimethylaminopropyl-3-ethylcarbodiimide (EDC) and 20 mM N-Hydroxysuccinimide (NHS) is prepared in 10 mM PBS. The SAM-coated substrate is then immediately immersed (EDC and NHS are not stable and will degrade within a few hours) for 2 hours at room temperature. This activates the surface for covalent conjugation to the protein of choice. Next, a 10 mM solution containing 0.01-0.1 mg/ml of IgG is added to the solution and incubated at room temperature for 2 hours. To block any remaining active sites, 100 mM ethanolamine is added and allowed to react for 20 minutes at room temperature. After washing thoroughly with PBS, the activated transducers are ready for use.

### Example 1a

### Bio-Nanosensor Detection of HIV Proteins

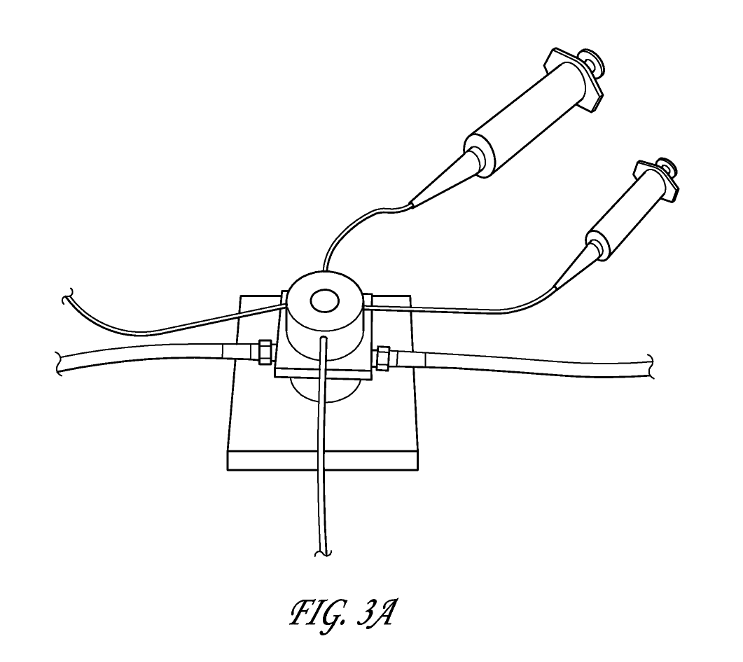

A bio-nanosensor HIV device was made and tested using a polyclonal sheep anti-HIV-1 gp120 (Aalto Bio Reagents Ltd, Dublin, Ireland), and a commercial preparation of gp120 (Aalto Bio Reagents) at a concentration of 0.2 mg/ml. The anti-gp120 was prepared following immunization with a conserved domain of 15 amino acids within HIV-1 envelope, and thus, recognizes conserved gp120 epitopes across all viruses isolated to date. This is an important feature, as it is well known that mutation within HIV-1 envelope protein gp120 is very common. To initiate the experiment a 5 mm diameter, 16 μm thick, 100 MHz quartz crystal with bonded 3 mm diameter gold electrodes made according to WO 2007/040566, “Method and Apparatus for Interfacial Sensing” by Ryszard M. Lec, corresponding to U.S. National Stage patent application Ser. No. 11/719,895, filed Feb. 21, 2008, was placed in a custom fabricated sensor holder, as shown in FIG. 3(A). This figure illustrates an embodiment of a bio-nanosensor device of the present invention—a prototypical bio-nanosensor chamber (bio-nanosensor is enclosed in the center window. A PC computer was used to control and collect data from a network analyzer (NA) (HP4395A), which drives the sensor and monitors the sensor response.

### Example 1b

### Bio-Nanosensor Monitoring

Following the sensor assembly, the bio-nanosensor was monitored continuously at a frequency of 100 MHz as a series of 5 μl blocking and sample solutions were placed in contact with the sensor, as shown in FIG. 3B. This figure illustrates the sensor resonant frequency change following steps of the immobilization process and sensor exposure to gp120. Reference measurements were first taken using TRIS buffer (starting measurement). Antibody solution (anti-gp120 at 1 mg/ml) was then added and incubated for 60 minutes to allow for antibody binding to the sensor surface using a standard chemiadsorption procedure (note initial negative resonance deflection). The antibody-coated sensor was then gently rinsed with TRIS buffer, followed by 15 -minute incubation in PBS to again gain reference measurements. Bovine Serum Albumin (BSA) (1 mg/ml) was then allowed to adsorb onto the sensor surface for 1 hour to block any remaining binding sites. The residual BSA was washed off in PBS, and then the sensor was probed by the addition of 5 μl of antigen (gp120) at 0.2 mg/ml (1 μg total protein), and monitored over the next 60 min. The bio-nanosensor response was consistently observed at 5-8,000 Hz (FIG. 3B), versus control (not shown).

### Example 1c

### Sensitivity of Bio-Nanosensors to gp120 Detection

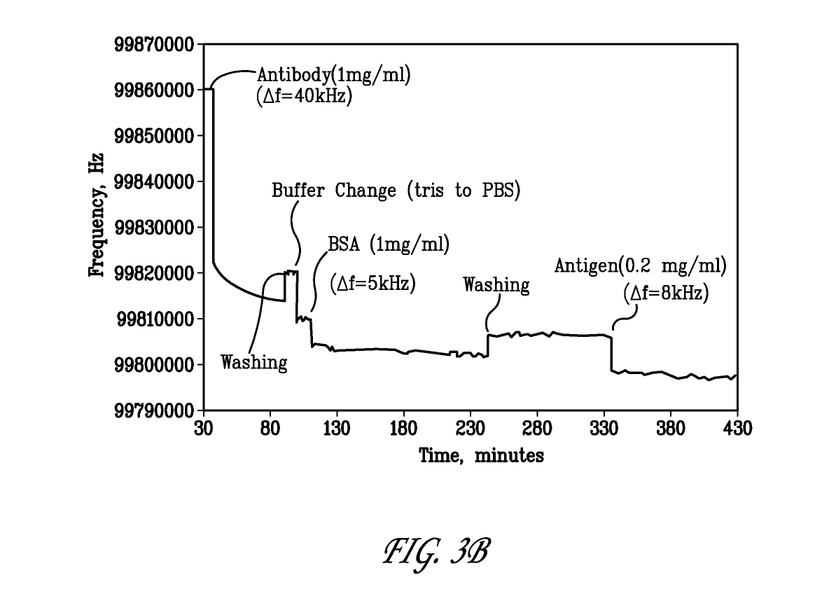

The sensitivity of gp120 detection was next determined by the prototype sensor by measuring responses to a range of gp120 concentrations (0.1-0.4 mg/ml). FIG. 4 illustrates the frequency response following the addition of gp120 to the bio-nanosensor coated with anti-gp120. The resulting sensitivity curve shown in FIG. 4 predicts a current lower limit of 25 nanograms per microliter (“ng/ul”). These data were obtained using a broad-band low sensitivity measurement system; accordingly, and as outlined below, it can be predicted that the sensitivity range of bio-nanosensors according to the present invention can be readily decreased into the range of from about 5 picograms (pg) to about 50 pg of adsorbed protein antigen. The versatility of the bio-nanosensor in detecting HIV proteins was verified by measuring HIV p24 levels using a similar approach. In this instance the bio-nanosensor was coated with rabbit polyclonal anti-p24 antibody (PerkinElmer, Boston, Mass. at 5 mg/ml) and then probed with p24 (PerkinElmer) at various clinically relevant concentrations (0-200 ng/ml). Similar results to those shown with gp120 were also observed (not shown).

### Example 2

### Bio-Nanosensor Detection of HIV Virions

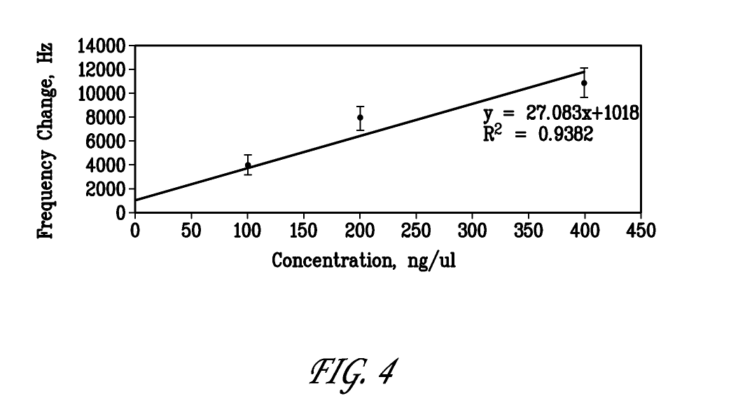

Using the basic principles outlined above for gp120 binding by the bio-nanosensor was applied for detecting HIV-1 in blood, and HIV-1 binding was directly analyzed using the bio-nanosensor. So that these preliminary experiments could be conducted without BSL2 restriction, a replication defective HIV-1 that was pseudotyped with or without gp120 was constructed. The backbone of this virus is HIV-1 NL43, which was rendered defective both in Env and Vpr genes. The negative control was thus a naïve NL43 virus; the test sample was NL43 virus that was pseudotyped with the R5/X4 dual tropic Env isolated from HIV-1 R3A (obtained from the University of Pennsylvania Center for AIDS Research, Philadelphia, Pa.). bio-nanosensor assays were conducted as described above, using sensors coated with polyclonal anti-gp120, blocked with BSA, washed, and then probed with either pseudotyped R3A or the NL43 alone (both at 78 ng/ml). Results of the resonant frequency shift seen in four separate experiments were pooled and are shown in histogram form in FIG. 5. This figure illustrates the bio-nanosensor resonant frequency change in response to HIV Negative Control-NL43 virus. Positive Control-R3A virus pseudotyped with R5/X4 ENV. A very slight response was seen with the NL43 virus negative control. In contrast, a significant shift of approximately 5,200 Hz was observed upon addition of the pseudotyped R3A that contained R5/X4 gp120 envelope protein.

Results. The results of these studies demonstrate that the bio-nanosensor technology is capable of detecting nanogram concentrations of HIV proteins gp120 and p24. Importantly, it has been demonstrated that bio-nanosensor can detect the presence of intact HIV-1 virus when using antibody probes. Accordingly, the bio-nanosensors and methods according to the present invention can be incorporated into portable devices capable of directly detecting HIV in blood. These results also point to the broader utility of bio-nanosensor in sensing other blood metabolites for which antibody or other binding reagents exist, such as insulin. In this regard the bio-nanosensors and methods can be used in reliable and portable HIV-1 diagnostic systems that enable sensing of multiple co-disease biomarkers. While the benefit of this technology will ultimately be widespread, the most beneficial results are in the application to less mobile populations, such as the elderly, and in settings that are remote to well equipped health clinics both here in the states and throughout the world.

### Example 3

### Thickness Shear Mode (TSM) Transducers

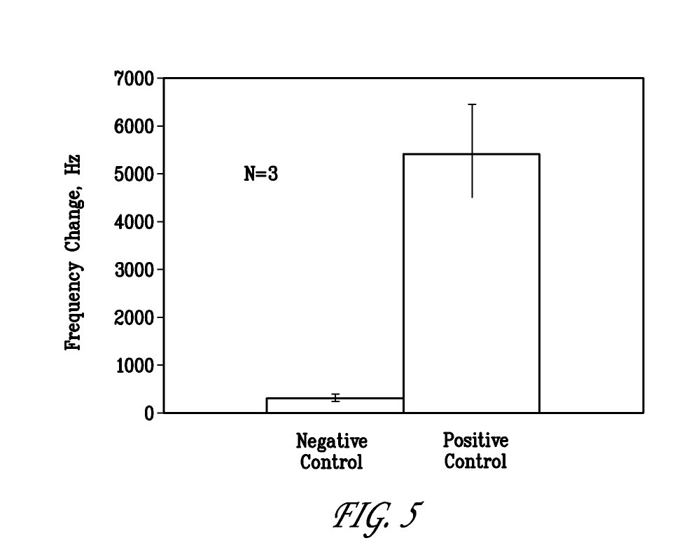

Suitable thickness shear mode (TSM) sensing microstructures are illustrated in FIG. 6, which provides (a) an embodiment of the bio-nanosensor device of the present invention—a single TSM sensor, and (b) an embodiment of the bio-nanosensor device of the present invention comprising a plurality of TSM sensors—a TSM sensor array; (c) an image of suitable 5 and 100 MHz TSM sensors that can be incorporated in the bio-nanosensors. FIG. 6(b) illustrates how a plurality of TSMs can be incorporated on different regions of a single piezoelectric crystal substrate.

### Example 4

### System Designs of Bio-Nanosensor Devices

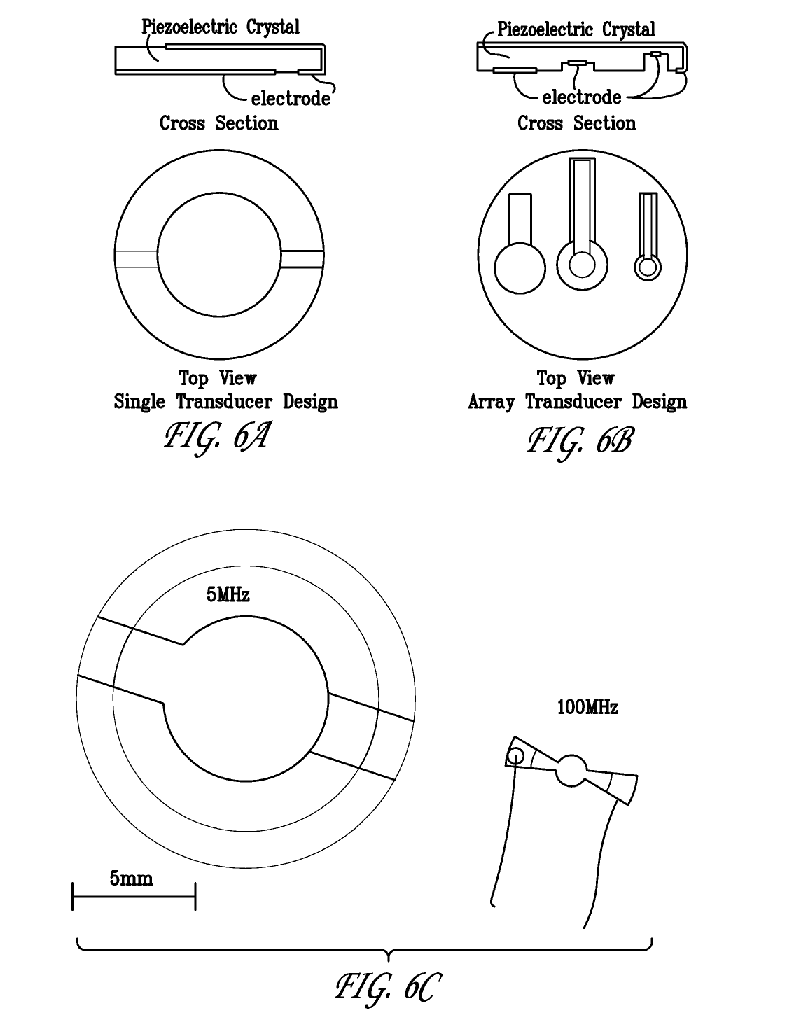

Suitable bio-nanosensors can be designed to include a fluidic chamber and an accompanying electronic measurement system. A measurement chamber can include one or more TSM sensors. A single TSM sensor can be used to measure one or more biomarkers, typically one biomarker, and an array or plurality of TSM sensors can be used for simultaneous detection of multiple biomarkers. Suitable bio-nanosensors will typically also include a sample delivery (fluidic) system, and a compartment for electronic circuitry and electrical connections as illustrated in FIG. 7. This figure illustrates an embodiment of a bio-nanosensor device of the present invention—HIV/AIDS Biochip Test Strip comprising (A) a disposable test strip complete with packaged reagents and electrical connections, and (B) an array of a plurality of TSM sensors and display module.

### Example 5

### Electronic Measurement Systems

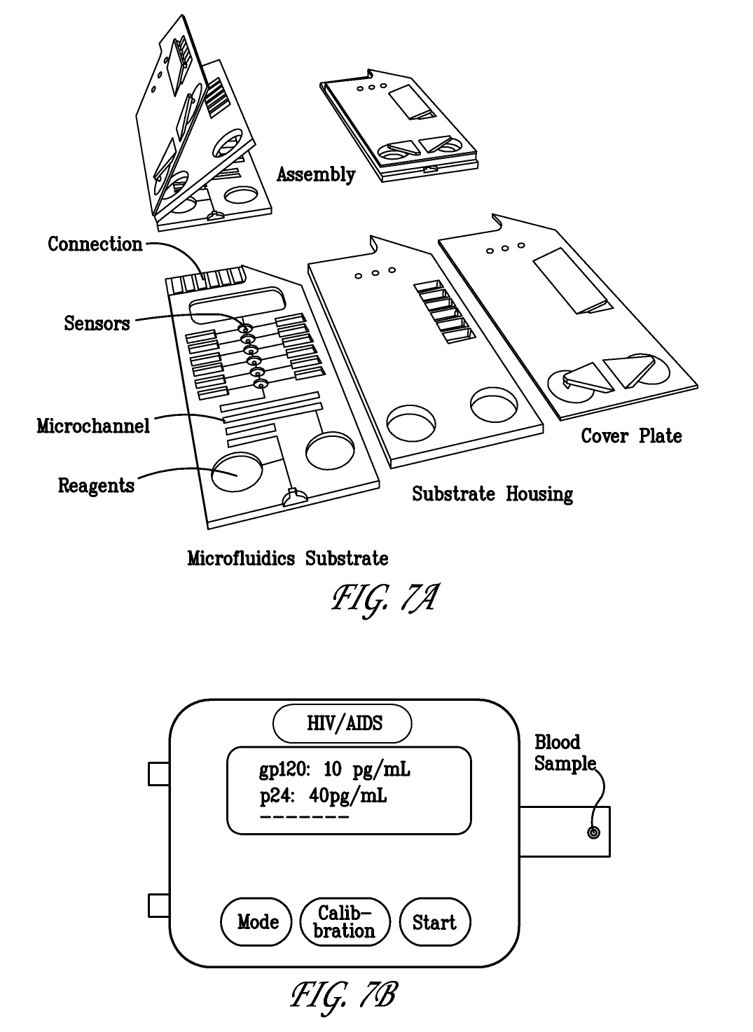

A suitable electronic measurement system can use a Network Analyzer (NA) technique and a personal computer (PC) for data acquisition and signal processing as shown in FIG. 8. This figure illustrates a suitable rapid broad frequency range (RBFR) electronic measurement system. The main feature of this technique is that the principle of operation involves the measurement of the trans-impedance of the TSM sensors. The Network Analyzer-based method provides a versatile measurement system: it allows for a rapid and wide frequency band scanning of the trans-impedance characteristics of the TSM sensor. The time and frequency domain signatures of the TSM response to antibody-antigen interactions and the time characteristics (kinetics) can be obtained easily. The TSM sensors is measured as a one-port or two-port device depending on the specific biological measurement requirements. All sensors with their enclosures (the chamber, reference liquid, cables, etc.) are calibrated in order to eliminate the influence of ambient conditions on the results. The measured sensor parameters that are used for data processing and subsequent biological interpretation include the sensor resonant frequency, magnitude, phase, impedance, and their signatures in the time domain. For the sensor array, a system of electronically controlled microwave switches can be used to change between different TSM sensors. However, in practical applications portable systems are preferable. Suitable electronic systems can be based on oscillatory circuitry as well phase-lock loop circuitry.

### Example 6

### Performance Testing and Optimization

Analysis of enhanced bio-nanosensor performance characteristics is conducted essentially as described above. Modifications to the sensor hardware, electronics and coupling described above are assessed using the polyclonal anti-gp120-gp120 detection system as shown above in FIGS. 3-4. Assay sensitivity is assessed over a range of gp120 concentrations (1 pg-100 ng). Once sensitivity is reliably obtained in the picogram range, analysis is extended to the detection of HIV-1 virions. As described above for FIG. 5, polyclonal anti-gp120 antibody is utilized as the surface agent interface, with R3A ENV+ and ENV-pseudotyped viruses as the test probes. This system thus includes an inherent gp120 (ENV) control and is applied over a range of virus concentrations from 1 pg-100 ng.

The immobilization protocol is used which results in sensitive and specific detection of biomarkers and virus while maintaining a stable and robust interface. In certain embodiments, the bio-nanosensors have the ability to identify, at a minimum, 60 pg/ml of targeted biomarkers (gp120, p24 and virion particles), which will establish this assay as equivalent in sensitivity to existing commercial lab tests. Immobilization of the antibodies and/or biomarker-specific ligands can be made even more robust by use of Protein G as a cross-linker at the bio-functionalized sensing interface.

TSM sensors with higher fundamental frequencies may be used, as the mass sensitivity increases with the square of operating frequency; a ring-type actuator can be integrated with the TSM sensor in order to concentrate the biomarkers on the surface of the TSM and accelerate the kinetics of the biding.

### Example 7

### Design and Development of a Bio-Nanosensor Assay for Multiple Biomarkers Associated with HIV/AIDS

The bio-nanosensor assay can be applied to measure HIV-1 directly in patient serum. A high-throughput piezoelectric bio-nanosensor incorporating multiple sensing elements on a single sensor substrate can also be used. As described above, the initial set of biomarkers are judiciously chosen to provide a unique HIV/AIDS patient health profile using only a few microliters of blood. HIV/AIDS and healthy control composite serum samples are screened in order to evaluate the performance of such an assay. A total of at least 100 individual patient samples and control donor samples can be screened using the bio-nanosensors of the present invention for measuring the concentration of HIV-1 levels and other biomarkers. This data is used to generate sensitivity and specificity data for each of the targets. Once accomplished, these assays can be incorporated onto a single, multi-chambered platform for simultaneous screening of biomarkers. This panel can be adapted for any biomarker where there is an antibody or other ligand to utilize on the interface surface. Thus, bio-nanosensors of the present invention can be used for HIV-1 detection, as well as for co-measurement of multiple disease markers that preferentially afflict HIV/AIDS patients.

### Example 8

### Bio-Nanosensor Analysis of HIV-1 and Disease Biomarkers in Patient Serum

The detection of HIV-1 in serum samples is accomplished using the parameters and conditions defined above. Human sera isolated from the peripheral blood of normal donors, and peripheral blood of HIV-1 isolated from infected patients is used. A small panel (10 each) of normal (uninfected) and HIV+ samples is tested to ensure assay sensitivity and specificity, using both anti-gp120, and independently, anti-p24 as antigens immobilized on the bio-functionalized sensing interface of a bio-nanosensor. Background noise due to non-specific serum binding to the sensor is determined in the analysis of HIV negative samples, and if present, can be overcome by blocking the sensor interface with normal serum prior to testing HIV positive control samples. Sera isolated from 25 normal donors and 25 HIV infected asymptomatic and AIDS patients is then tested. The results of this analysis is compared to standard laboratory tests using the p24 ELISA, as described above.

### Example 9a

### Detection of Additional Disease-Linked Biomarkers

The bio-nanosensors of the present invention can also be used for the simultaneous detection of multiple disease-associated biomarkers. Accordingly, the antibodies or ligands indicative of additional infectious agents other than HIV, and other physiological biomarkers to normal and disease states can be detected using the bio-sensors of the present invention to detect the presence of: cells, for example CD4; proteins, lipids and other biomarkers, for example insulin, C-peptide, IL-6, HbA1C, Hb (hemoglobin), creatinine, Erythropoietin (EPO), AST, ALT, Biliribin, LDH, GGT and AP; and AP, antibodies against or molecular components of viruses, bacteria, fungi, protozoans and parasites, for example caused hepatitis (HV) A, B, C, D and E, antibodies against herpes simplex virus (HSV), cytomegalovirus (CMV) and Epstein-Barr virus (EBV), or any combination thereof.

For this analysis, markers of insulin resistance and/or diabetes are chosen to exemplify the detection of additional disease-linked biomarkers in view of the metabolic implications of HIV/AIDS, HAART treatment, and association with aging. The particular biomarkers against insulin resistance and/or diabetes are listed in Table I. Specifically, they include HbA1c (glycosylated hemoglobin A1c), fasting plasma insulin, and C-peptide levels: all of which rise significantly in insulin resistance and/or diabetes patients; and Interleukin-6 (IL-6), which induces insulin resistance and is elevated in serum from patients with these syndromes. Additionally, fasting plasma glucose is evaluated as a control factor (each serum sample is tested by gluco-test).

TABLE I
HIV/AIDS - Disease Biomarker Characteristics

| Biomarkers used for | | | | | |
|---|---|---|---|---|---|
| HIV/AIDS patient | Molecular | | Clinical | Clinical Relevant | |
| health profile | Weight | | Relevance | Concentration | |
| gp120 | 120 | kDa | Presence of this | >60 | pg/ml |
| | | | antigen in serum | | |
| | | | confirms infection | | |
| p24 | 24 | kDa | Presence of this | >60 | pg/ml |
| | | | antigen in serum | | |
| | | | confirms infection | | |
| Insulin | 5808 | Da | Elevated in | >10 | μU/ml |
| | | | diabetes | | |
| HbA1C | 18 | kDa | Elevated in | >6% | |
| | | | diabetes | | |
| C-peptide | 3020 | Da | Elevated in | >12 | ng/ml |
| | | | diabetes | | |
| IL-6 | 28 | kDa | Elevated in | >6 | ng/ml |
| | | | diabetes | | |

### Example 9b

### Health Profiles of Patients with and Without HIV

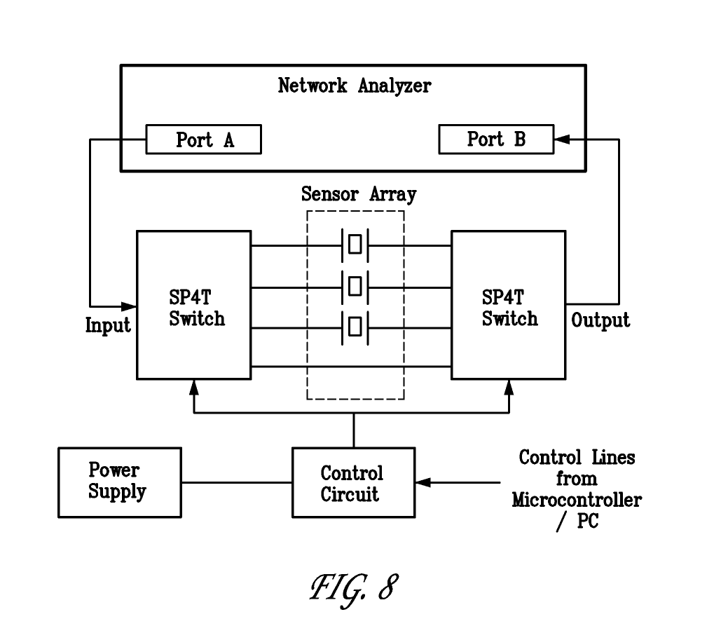

For the purposes of these studies, archived serum samples are analyzed from the University of Pennsylvania Center for AIDS Research serum bank, which have specific health profiles in hand; i.e., HIV/AIDS status (virus and CD4 levels), insulin resistance and diabetes. Initial experiments are conducted on each biomarker individually to ensure the specificity and detection sensitivity. The procedure employed is identical to that described above for gp120, as antibody is available to the human component of each of these markers. Once the assays are established for each of these biomarkers, samples from the CFAR serum bank are analyzed in a blinded manner and the panel of biomarker expression compiled. A hypothetical readout for these studies is shown in FIG. 9, the expected distribution (health profile) of HIV/AIDS patients with insulin resistance and diabetes: Patient 1 provides an example of an individual successfully treated by HAART with low viral load and normal CD4 values, and in this instance all of the insulin resistance markers are within normal limits. In contrast, Patient 2 displays a high viral load, low CD4 levels and increased insulin resistance markers.

### Example 9c

### A Multi-Functional Bio-Nanosensor

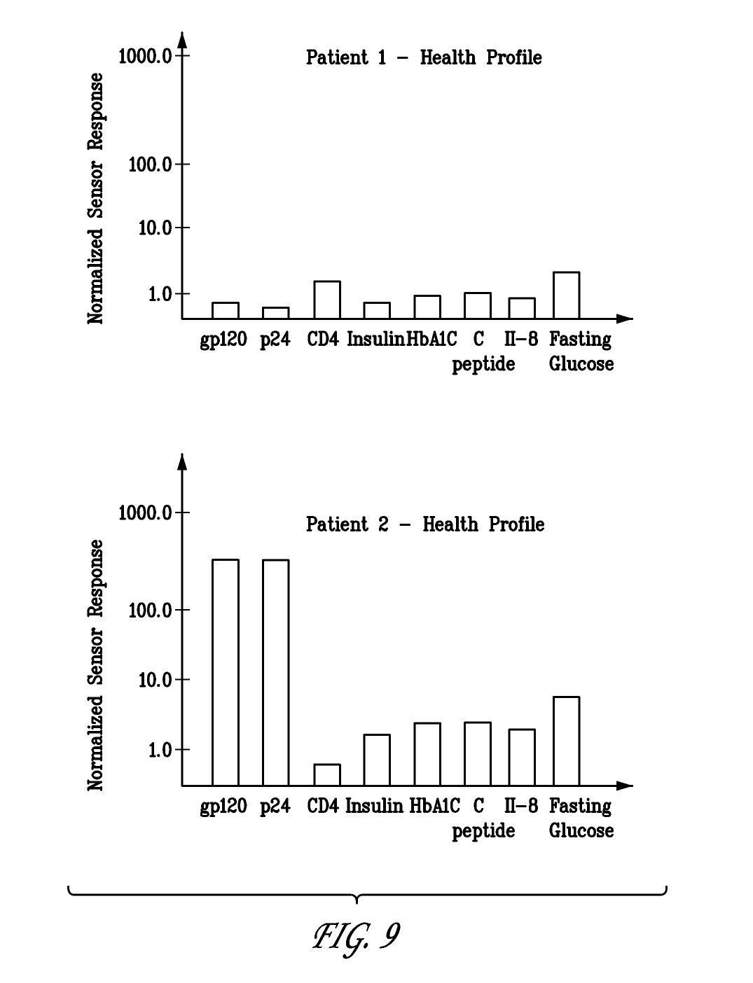

The fabrication process of the multifunctional bio-nanosensor will include three main steps. A description of how to carry out each of the individual steps outlined in FIG. 10 is described throughout this specification. Accordingly, FIG. 10 illustrates the steps for fabricating a multifunctional bio-nanosensor according to the present invention: design, process development and identification of packing density of sensing elements for determining the size of the chip.

Results. The results from the bio-nanosensor tests closely match those of the ELISA and will provide precise concentrations of biomarkers in the sera. The resulting data from these assays reveals important information about HIV/AIDS detection and accompanying health conditions. On more a fundamental level, patient screening helps to optimize a panel of chosen antibody-biomarker systems. Accordingly, the bio-nanosensors can be used as a useful tool for biomarker evaluation.

### Example 10

### Medical Management of Patients with HIV/AIDS Enabled by the Bio-Nanosensor Assay Technology

The devices and methods of the present invention give rise to a new form of diagnostic blood assay—the bio-nanosensor. The bio-nanosensor is robust enough to be used to determine the initial diagnosis of HIV-1, for the long-term management of HIV-1 virus load in response to HAART or other therapies, and for the co-monitoring of multiple disease based conditions using serum biomarkers. The bio-nanosensor devices are sensitive, specific, rapid, inexpensive and portable. The benefits of the devices of the present invention are extraordinary in terms of the application to in-home or other point-of-care settings that are remote from health clinics: because of this, the bio-nanosensor devices are particularly applicable to care of the elderly and to less-affluent settings and/or cultures.

The bio-nanosensor-based assays are able to provide a health profile of HIV/AIDS patients on demand, quickly and in convenient manner, at the doctor office or at the patient home. A friendly user procedure for collecting small sample of blood and suitable display of the results within a few minutes, which, in combination with accompanying standard diagnosis, will facilitate the overall HIV/AIDS treatment process, including a very important issue of optimization of the treatment tailored for individual health needs of the patient. In addition, having a recorded an evolution of heath profile over long period of time will improve the outcome of the treatment and next, it should lead to better understanding of the HIV/AIDS. The bio-nanosensors and methods disclosed herein will advance HIV/AIDS diagnosis and treatment and give rise to a significant breakthrough management of HIV/AIDS patients.

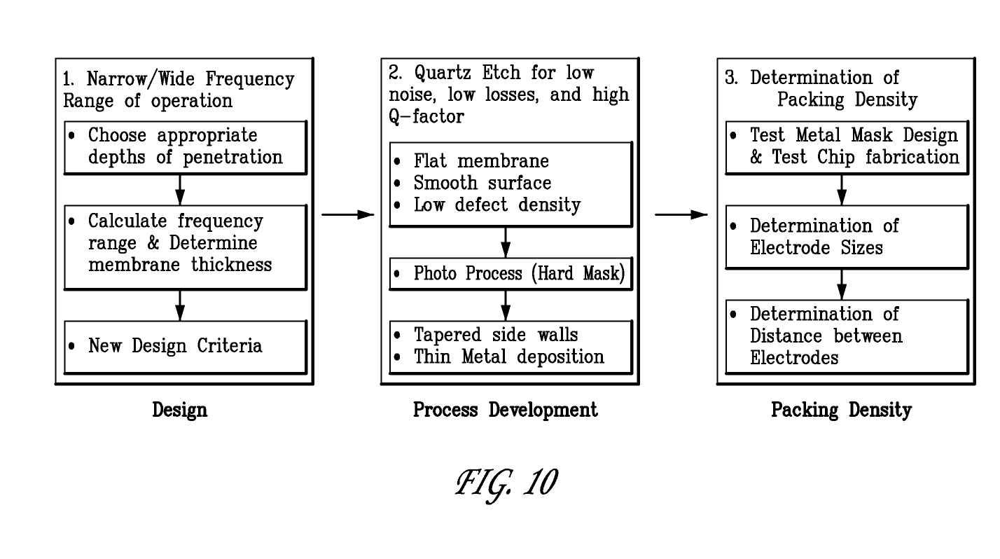

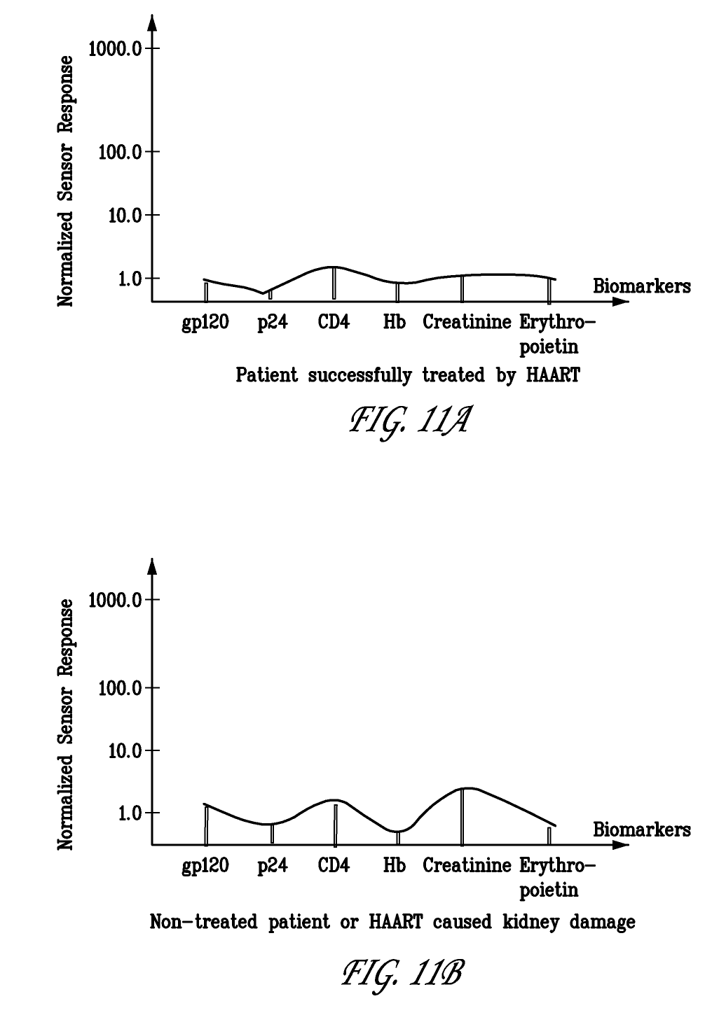

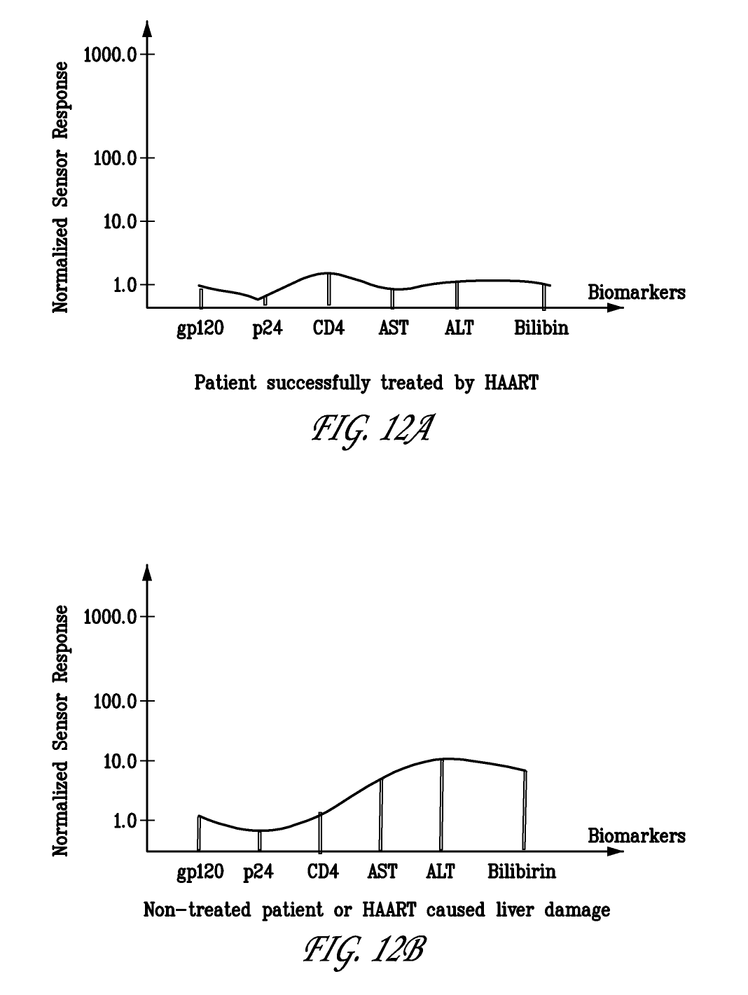

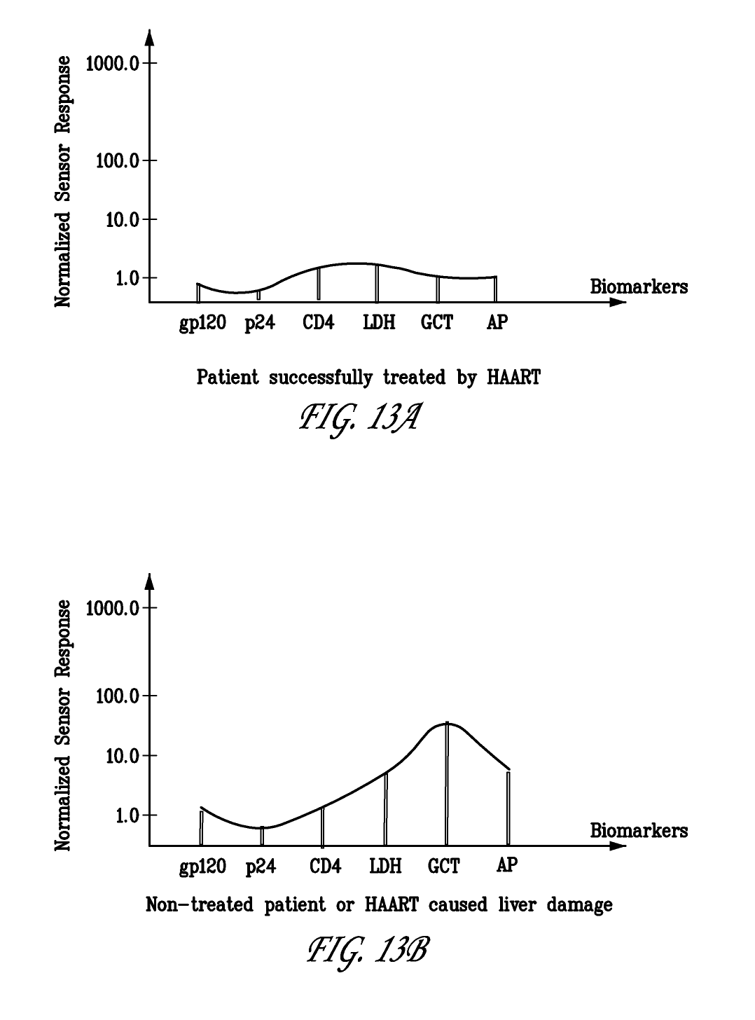

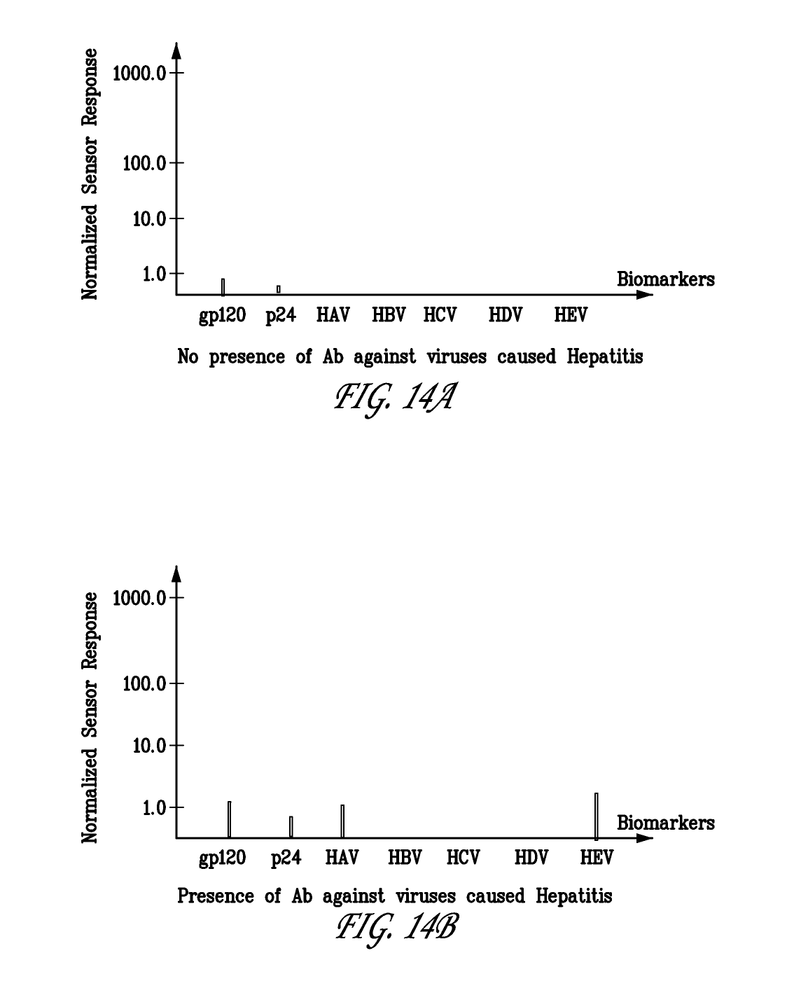

A typical patient's readouts are shown in FIGS. 11-15. FIG. 11 illustrates the bio-nanosensor profile results using the devices and methods of the present invention that can be attained from HIV/AIDS patients with anemia and kidney involvement. FIG. 12 illustrates the bio-nanosensor profile results using the devices and methods of the present invention that can be attained from HIV/AIDS patients with liver involvement I. FIG. 13 illustrates the bio-nanosensor profile results using the devices and methods of the present invention that can be attained from HIV/AIDS patients with liver involvement II. Patient 1 (A) provides an example of an individual successfully treated by HAART with low viral load and normal CD4 values, normal Hemoglobin value and in this instance all of the liver and kidney markers are within normal limits. In contrast, Patient 2 (B) displays a high viral load, low CD4 levels and abnormal values for those markers. Additionally FIGS. 14 and 15 show the example of secondary viral infection (B) during progress of HIV/AIDS disease. FIG. 14 illustrates the bio-nanosensor profile results using the devices and methods of the present invention that can be attained from HIV/AIDS patients with secondary viral infection I. FIG. 15 illustrates the bio-nanosensor profile results using the devices and methods of the present invention that can be attained from HIV/AIDS patients with secondary viral infection II.

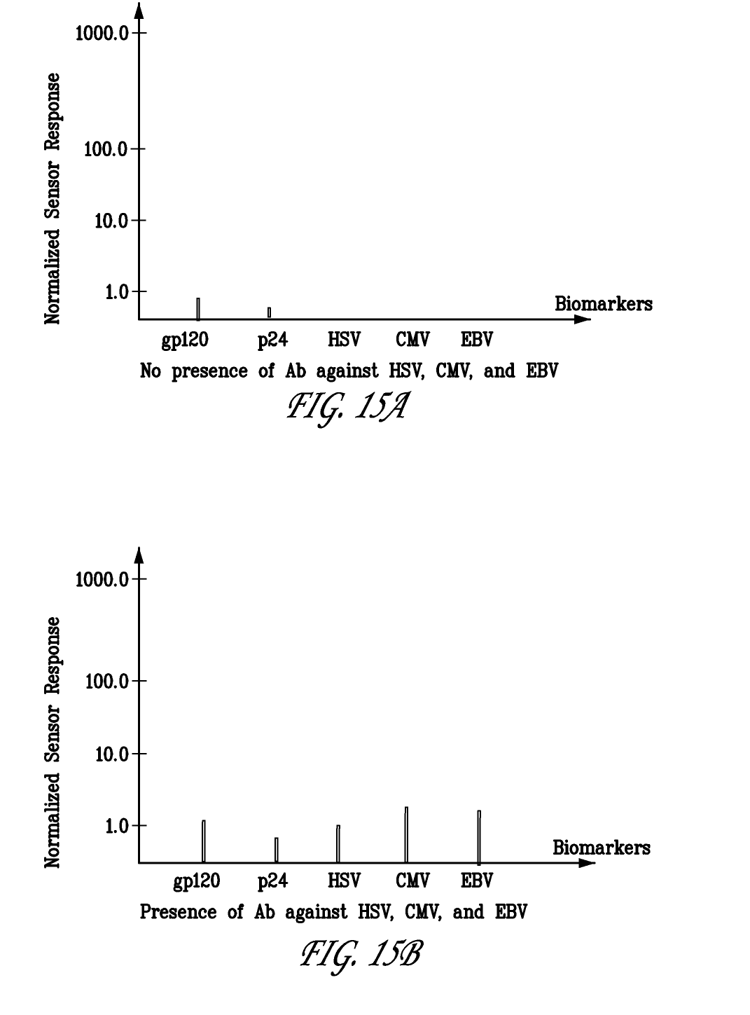

FIG. 16 illustrates the response of bio-nano-sensor (BNS) analysis on blood plasma obtained from HIV-1 negative and positive blood samples. Here we directly evaluated the utility of the BNS assay approach by measuring for the presence of HIV particles in plasma obtained from infected HIV/AIDS patients (positive control n=10) with the plasma obtained from normal uninfected healthy volunteers (negative control n=10). We used a thickness shear mode BNS operating at 200 MHz fundamental frequency. These results show that at 200 MHz the BNS device was able to successfully detect HIV-1 in the whole blood plasma from infected subjects, and that the level of detection was statistically distinct from the background response observed in the uninfected normal blood plasma.

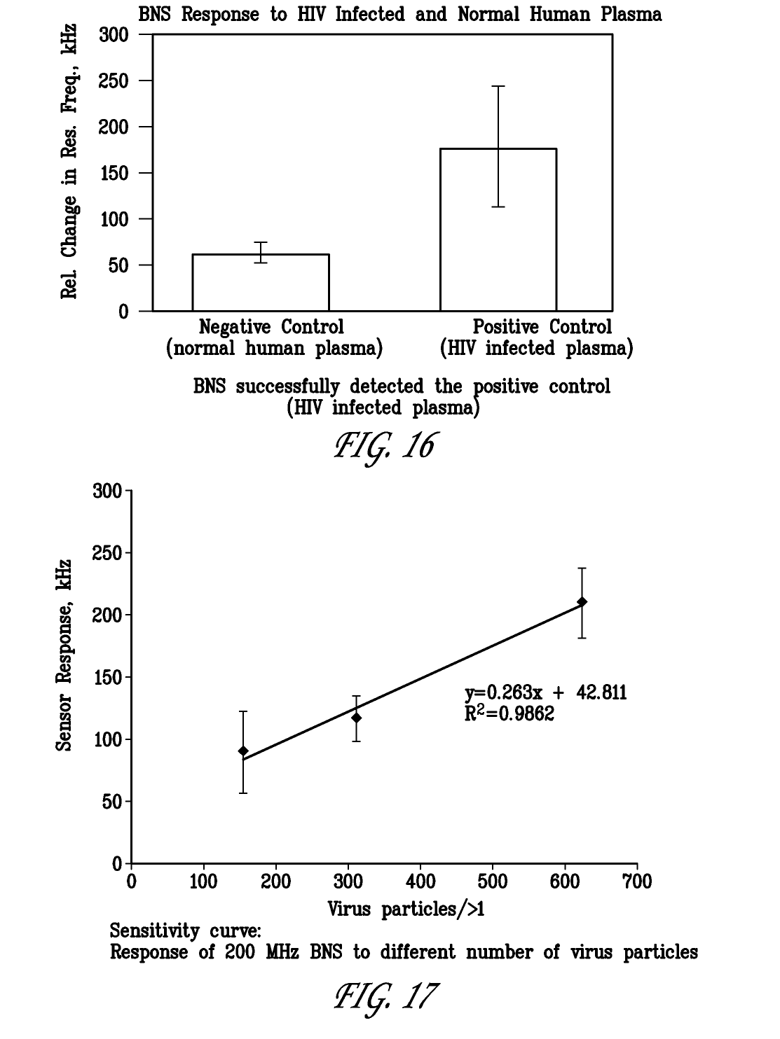

FIG. 17 illustrates the sensitivity characteristics of the BNS device to various concentrations of HIV-1 virus particles. We measured the response at 200 MHz of the BNS to a linear range of virus particles/μl obtained from HIV/AIDS patients. As can be seen from the experimental data presented, at 200 MHz the BNS is capable of detecting ˜150 virus particles/μl. Importantly, a linear increase in sensor response with the number of virus particles is also observed. The data shows a very strong correlation coefficient R²=0.9862 to linear fit. Thus, the equation describing the sensor sensitivity (straight line) can be used to determine the number of virus particles in any sample containing the unknown number of viral particles, collected from a patient.

The present inventions provide devices and methods for enabling an intervention model directed toward the coordination and simplification of HIV treatment, particularly in the elderly who often suffer from multiple medical conditions. Accordingly, the bio-nanosensor technology described herein is capable of direct detection of the presence of HIV in blood along with other factors/markers associated with conditions that commonly afflict patients on HAART, such as elderly patients. Configured in portable and disposable devices that can be used at the point-of-care, the disclosed bio-nanosensor technology is designed to better manage the medical conditions of patients with HIV/AIDS. The disclosed inventions will enable patients and health professionals to track HIV infection along with other related conditions on a real-time basis. Through direct diagnostic feedback, the bio-nanosensor device will lead to the coordination of HAART with therapies directed toward other conditions, such as insulin resistance and diabetes. The disclosed inventions are capable of reducing disease monitoring to a single instrument that does not require disjointed laboratory procedures this technology may dramatically improve the medical treatment as well as quality of life outcome in elderly patients with HIV/AIDS and other conditions.

Although the present invention has been described in relation to particular embodiments thereof, many other variations and modifications and other uses is apparent to those skilled in the art. It is preferred, therefore, that the present invention be delineated not by the specific disclosure herein, but only by the appended claims.

What is claimed:

1. A bio-nanosensor device, comprising:
a thickness shear mode transducer comprising:
a piezoelectric crystal characterized whereby an applied alternating electrical voltage induces an oscillating shear mechanical strain over a broad frequency range, and
whereby the thickness shear mode transducer is capable of producing a standing acoustic wave within the piezoelectric crystal, the thickness shear mode transducer being capable of transmitting a shear wave into a biofluid adjacent to a bio-functionalized sensing interface of the piezoelectric crystal to give rise to a resonant acoustic wave frequency change measurable by said biosensor device;
wherein the bio-functionalized sensing interface comprising one or more of the following antibodies: anti-gp120, anti-p24 and anti-CD4, anti gp41, anti-gp160, ati-V3, anti Gag-anti p24, anti Nef, anti-Pol and Rev-anti RT, anti-IN, anti-Tat, anti-Vif, anti-Vpx, anti-p27, anti p17, anti-Nef, anti-Pol-anti-protease, anti-integrase anti-Vpr, anti-Vpu, and anti-CD4, wherein the antibodies are immobilized at the bio-functionalized sensing interface;
a fluidic chamber capable of containing said biofluid, the fluidic chamber comprising one or more fluidic conduits capable of fluidicly communicating at least one fluid; and
one or more electrical leads in electrical communication with one or more electrodes mounted directly adjacent to said piezoelectric crystal and said bio-functionalized sensing interface.

2. The bio-nanosensor device of claim 1, wherein the one or more fluidic conduits are capable of fluidicly communicating at least one fluid comprising a washing fluid, a blocking agent, a buffer, a biomarker, an antibody, a biofluid, an antigen, a coupling agent, a wetting agent, a cleaning agent, or any combination thereof.

3. The bio-nanosensor of claim 2, wherein the blocking agent comprises BSA and TRIS.

4. The bio-nanosensor device of claim 1, further comprising one or more additional biomarker-sensing ligands specific to one or more biomarkers for monitoring the presence of one or more additional disease states other than HIV/AIDS, the biomarker-sensing ligands immobilized at the bio-functionalized sensing interface.

5. The bio-nanosensor device of claim 1, comprising a plurality of said thickness shear mode transducers, at least one of said thickness shear mode transducers comprising an antibody or biomarker-sensing ligand immobilized at its bio-functionalized sensing interface different than at least one other of the biomarker-sensing ligands of another thickness shear mode transducer.

6. The bio-nanosensor device of claim 1, capable of detecting the presence of HIV virus in no more than 10 μl of blood in less than 15 minutes.

7. A method of determining the presence of HIV virus in a biofluid, comprising:
contacting a biofluid suspected of comprising HIV to a bio-functionalized sensing interface comprising one or more of the following antibodies and ligands: anti-gp120, anti-p24, anti gp41, anti-gp160, ati-V3, anti Gag-anti p24, anti Nef, anti-Pol and Rev-anti RT, anti-IN, anti-Tat, anti-Vif, anti-Vpx, anti-p27, anti p17, anti-Nef, anti-Pol-anti-protease, anti-integrase anti-Vpr, anti-Vpu, and anti-CD4, wherein the antibodies being immobilized at the bio-functionalized sensing interface, the bio-functionalized sensing interface being coupled to a piezoelectric crystal:
inducing an oscillating shear mechanical strain of the piezoelectric crystal to give rise to a shear wave being transmitted into the biofluid adjacent to the bio-functionalized sensing interface of the piezoelectric crystal;
measuring the frequency of the standing acoustic wave of the piezoelectric crystal; and
correlating the frequency of the standing acoustic wave to the presence of HIV virus in the biofluid.

8. The method of claim 7, wherein the biofluid comprises blood, and the presence of HIV virus is detected using no more than 10 μl of blood in less than 15 minutes.

9. The method of claim 7, further comprising the steps of:
contacting a control fluid not comprising at least one of the following: HIV virus, gp120, p24, anti gp41, anti-gp160, ati-V3, anti Gag-anti p24, anti Nef, anti-Pol and Rev-anti RT, anti-IN, anti-Tat, anti-Vif, anti-Vpx, anti-p27, anti p17, anti-Nef, anti-Pol-anti-protease, anti-integrase anti-Vpr, anti-Vpu, and anti-CD4, to the bio-functionalized sensing interface;
inducing an oscillating shear mechanical strain of the piezoelectric crystal to give rise to a shear wave being transmitted into the control fluid adjacent to the bio-functionalized sensing interface of the piezoelectric crystal;
measuring the frequency of a standing acoustic wave of the piezoelectric crystal arising from the shear wave being transmitted into the control fluid; and
correlating the difference between the frequency of the standing acoustic wave measured with the control fluid to the frequency of the standing acoustic wave measured with the biofluid to the presence of HIV virus in the biofluid.

10. The method of claim 9, wherein the difference between the frequency of the standing acoustic wave measured with the control fluid to the frequency of the standing acoustic wave measured with the biofluid is correlated to the concentration of HIV virus in the biofluid.

11. The method of claim 9, wherein the method further detects one or more of the following:
antibodies or ligands for additional infectious agents other than HIV;
physiological biomarkers indicative of normal or disease states, proteins, lipids, biomarkers; and
antibodies against, or molecular components of, one or more of the following: viruses, bacteria, fungi, protozoans and parasites.

12. The method of claim 7, wherein the bio-functionalized sensing interface further comprises one or more additional biomarker-sensing ligands specific to one or more biomarkers for monitoring the presence of one or more additional disease states other than HIV/AIDS, wherein the biomarker-sensing ligands are immobilized at the bio-functionalized sensing interface.

13. The method of claim 7, wherein the biofluid is contacted with the bio-functionalized sensing interface in a fluidic chamber, the fluidic chamber comprising one or more fluidic conduits capable of fluidicly communicating at least one or more of the following fluids into the fluidic chamber: a washing fluid, a blocking agent, a buffer, a biomarker, an antibody, a biofluid, an antigen, a coupling agent, a wetting agent, a cleaning agent.

14. The method of claim 7, wherein the frequency of the standing acoustic wave is correlated to the concentration of HIV virus in the biofluid.

15. The method of claim 7, wherein the biofluid is contacted to a plurality of bio-functionalized sensing interfaces, each bio-fluid contacting surface comprising an antibody or biomarker-sensing ligand attached thereto, the antibodies or biomarker-sensing ligands immobilized at one of the bio-functionalized sensing interfaces being different than the antibodies or biomarker-sensing ligands immobilized at one or more of the other bio-functionalized sensing interfaces.

16. The method of claim 7, wherein each of the antibodies or biomarker-sensing ligands immobilized at each of the bio-functionalized sensing interfaces are different.

17. A method for monitoring the progress of therapy or of vaccine prevention of a patient having HIV virus, comprising:
obtaining a biofluid specimen from the patient;
contacting the biofluid specimen to a bio-functionalized sensing interface comprising one or more of the following antibodies: anti-gp120, anti-p24, anti gp41, anti-gp160, ati-V3, anti Gag-anti p24, anti Nef, anti-Pol and Rev-anti RT, anti-IN, anti-Tat, anti-Vif, anti-Vpx, anti-p27, anti p17, anti-Nef, anti-Pol-anti-protease, anti-integrase anti-Vpr, anti-Vpu, and anti-CD4, the antibodies being immobilized at the bio-functionalized sensing interface, the bio-functionalized sensing interface being coupled to a piezoelectric crystal;
inducing an oscillating shear mechanical strain of the piezoelectric crystal to give rise to a shear wave being transmitted into the biofluid adjacent to the bio-functionalized sensing interface of the piezoelectric crystal;
measuring the frequency of the standing acoustic wave of the piezoelectric crystal; and
correlating the frequency of the standing acoustic wave to the concentration of HIV virus in the biofluid specimen.

18. The method of claim 17, wherein the biofluid specimen comprises blood, and the presence of HIV virus is detected using no more than about 10 μl of the blood specimen in less than about 15 minutes.

19. The method of claim 17, further comprising the steps of:
contacting a control fluid not comprising HIV virus, gp120, p24, anti gp41, anti-gp160, ati-V3, anti Gag-anti p24, anti Nef, anti-Pol and Rev-anti RT, anti-IN, anti-Tat, anti-Vif, anti-Vpx, anti-p27, anti p17, anti-Nef, anti-Pol-anti-protease, anti-integrase anti-Vpr, anti-Vpu, and anti-CD4, to the bio-functionalized sensing interface;
inducing an oscillating shear mechanical strain of the piezoelectric crystal to give rise to a shear wave being transmitted into the control fluid adjacent to the bio-functionalized sensing interface of the piezoelectric crystal;
measuring the frequency of a standing acoustic wave of the piezoelectric crystal arising from the shear wave being transmitted into the control fluid; and
correlating the difference between the frequency of the standing acoustic wave measured with the control fluid to the frequency of the standing acoustic wave measured with the biofluid to the presence of HIV virus in the biofluid.

20. The method of claim 19, wherein the difference between the frequency of the standing acoustic wave measured with the control fluid to the frequency of the standing acoustic wave measured with the biofluid is correlated to the concentration of HIV virus in the biofluid.

21. The method of claim 19, wherein the method further detects the presence of one or more of the following in the biofluid: antibodies or ligands for additional infectious agents other than HIV;
physiological biomarkers indicative of normal or disease states, proteins, lipids, biomarkers; and
antibodies against, or molecular components of, one or more of the following: viruses, bacteria, fungi, protozoans and parasites.

22. The method of claim 17, wherein the bio-functionalized sensing interface further comprises one or more additional biomarker-sensing ligands specific to one or more biomarkers for monitoring the presence of one or more additional disease states other than HIV/AIDS, the biomarker-sensing ligands immobilized at the bio-functionalized sensing interface.

23. The method of claim 17, wherein the biofluid is contacted with the bio-functionalized sensing interface in a fluidic chamber, the fluidic chamber comprising one or more fluidic conduits capable of fluidicly communicating at least one or more of the following fluids into the fluidic chamber: a washing fluid, a blocking agent, a buffer, a biomarker, an antibody, a biofluid, an antigen, a coupling agent, a wetting agent, a cleaning agent.

24. The method of claim 17, wherein the frequency of the standing acoustic wave is correlated to the concentration of HIV virus in the biofluid.

25. The method of claim 17, wherein the biofluid is contacted to a plurality of bio-functionalized sensing interfaces, each bio-fluid contacting surface comprising an antibody or biomarker-sensing ligand attached thereto, the antibodies or biomarker-sensing ligands immobilized at one of the bio-functionalized sensing interfaces being different than the antibodies or biomarker-sensing ligands immobilized at one or more of the other bio-functionalized sensing interfaces.

26. The method of claim 17, wherein each of the antibodies or biomarker-sensing ligands immobilized at each of the bio-functionalized sensing interfaces are different.

27. The method of claim 11, wherein the cells comprise CD4.

28. The method of claim 11, wherein the proteins, lipids and other biomarkers comprise one or more of the following: insulin, C-peptide, IL-6, HbA_{1C}, Hb (hemoglobin), creatinine, Erythropoietin (EPO), AST, ALT, Biliribin, LDH, GGT and AP.

29. The method of claim 11, wherein the viruses comprise one or more of the following: hepatitis (HV) A, B, C, D and E.

30. The method of claim 11, wherein the antibodies comprise an antibody against herpes simplex virus (HSV), an antibody against cytomegalovirus (CMV), or an antibody against Epstein-Barr virus (EBV), or any combination thereof.

31. The method of claim 21, wherein the cells comprise CD4.

32. The method of claim 21, wherein the proteins, lipids and other biomarkers comprise one or more of the following: insulin, C-peptide, IL-6, HbA_{1C}, Hb (hemoglobin), creatinine, Erythropoietin (EPO), AST, ALT, Biliribin, LDH, GGT and AP.

33. The method of claim 21, wherein the viruses comprise one or more of the following: hepatitis (HV) A, B, C, D and E.

34. The method of claim 21, wherein the antibodies comprise an antibody against herpes simplex virus (HSV), an antibody against cytomegalovirus (CMV), or an antibody against Epstein-Barr virus (EBV), or any combination thereof.
